(19) United States
(12) Patent Application Publication
(10) Pub. No.: US 2016/0158582 A1
(43) Pub. Date: Jun. 9, 2016

(54) PRODUCTS IN DISPENSER FOR OXIDATIVE CHANGE TO COLOUR OF KERATINOUS FIBRES

(71) Applicant: Henkel AG & Co. KGaA, Duesseldorf (DE)

(72) Inventors: Burkhard Mueller, Duesseldorf (DE); Norbert Schettiger, Hilden (DE); Constanze Neuba, Grevenbroich (DE)

(21) Appl. No.: 15/045,333

(22) Filed: Feb. 17, 2016

Related U.S. Application Data
(63) Continuation of application No. PCT/DE2014/200419, filed on Aug. 22, 2014.

(30) Foreign Application Priority Data
Aug. 27, 2013 (DE) 10 2013 217 024.6

(51) Int. Cl. A61Q 5/10; A61K 8/37; A61K 8/60; A61K 8/81; B65D 35/22; A61K 8/46; A61K 8/86; A61Q 5/08; B65D 81/32; B65D 83/14; A61K 8/34; A61K 8/73

(52) U.S. Cl. CPC A61Q 5/10; A61K 8/342; A61K 8/375; A61K 8/602; A61K 8/8158; A61K 8/73; A61K 8/463; A61K 8/86; A61Q 5/08; B65D 81/32; B65D 83/14; B65D 35/22; A61K 2800/48; A61K 2800/88; A61K 2800/87

(57) ABSTRACT

A product for the oxidative coloring and/or lightening of keratinic fibers includes (1) a dispenser, having two chambers (A) and (B) separate from each other, and having an outlet opening (C) which is in communication with chamber (A) and with chamber (B); (2) an aqueous cosmetic preparation (a) in chamber (A), having a viscosity V1 of 1000 to 100,000 mPas (22° C./Brookfield viscometer/spindle 5/4 rpm), and (3) an aqueous cosmetic preparation (b) in chamber (B), having a viscosity V2 of 1000 to 100,000 mPas (22° C./Brookfield viscometer/spindle 5/4 rpm), wherein the ratio of the viscosities V1/V2 has a value of 0.3 to 3.0.

### FIELD OF THE INVENTION

The present invention generally relates to a product for the coloring and/or lightening of keratinic fibers, which product includes a dispenser having two chambers separate from one another, whereby the chambers have a common outlet opening. Each of the two chambers includes a preparation with a special viscosity, and the viscosities of the two preparations are precisely matched to one another.

### BACKGROUND OF THE INVENTION

The changing of the color of keratinic fibers, particularly of hair, is an important field in modern cosmetics. As a result, the appearance of the hair can be adapted both to current fashion trends and to the individual wishes of the individual person. The skilled artisan is aware of various options for changing the color of hair.

Hair color can be changed temporarily by the use of direct dyes. In this case, already formed dyes diffuse from the coloring agent into the hair fiber. Dyeing with direct dyes is associated with less hair damage, but a disadvantage is the low durability and the rapid washing out of the colors obtained with direct dyes.

Oxidative color-changing agents are usually used if the consumer wants a long-lasting color result or a shade that is lighter than the consumer's original hair color. So-called oxidation coloring agents are used for permanent, intensive colors with suitable fastness properties. Such coloring agents typically include oxidation dye precursors, so-called developer components and coupler components, which together form the actual dyes under the influence of oxidizing agents, usually hydrogen peroxide. Oxidation coloring agents are characterized by excellent, long-lasting color results.

The mere lightening or bleaching of hair often occurs with the use of one or more oxidizing agents without the addition of oxidation dye precursors. Hydrogen peroxide is usually used in this case as the oxidizing agent.

Oxidative color-changing agents are typically found on the market in the form of two-component agents, in which the two components are packaged separately in two separate containers and must be mixed together shortly before use.

The first component is usually a formulation, which has been made alkaline and is often available in the form of a cream or a gel and which, provided that a change in color as well is desired concurrently with the lightening, also includes in addition oxidation dye precursors. This first component is provided in most cases in a tube, less often in a plastic or glass container.

The second component is a formulation, which has usually been made acidic for reasons of stability and which includes hydrogen peroxide in concentrations of 3 to 12% by weight as the oxidizing agent. The oxidizing agent formulation mostly has the form of an emulsion or dispersion and as a rule is made available in a plastic bottle with a re-closable outlet opening (developer bottle).

To prepare the ready-to-use mixture, the consumer must mix the two components together shortly before use. For this purpose, normally the alkaline cream or gel component must be transferred completely from the tube or glass or plastic container into the developer bottle; then both components are mixed together as completely and homogeneously as possible by shaking, and finally removed via an outlet opening in the top of the developer bottler.

This separate mixing operation has a number of drawbacks for consumers, however. Thus, the quantity ratio of the two components can be changed by the incomplete emptying of the tube; this results in variations in the desired color result. If the shaking or mixing of the two components is too short, the application mixture is not homogeneous with the result of a non-uniform color result. Moreover, it is also desirable for reasons of ease of use to eliminate this additional mixing step entirely.

To avoid these drawbacks, multi-chamber containers (dispensers) with a common discharge opening were developed, in which the two components are mixed in the discharge device of the dispenser during the discharging. The removing of the application mixture via the dispenser makes the mixing of the components by the consumer superfluous and has greatly increased the ease of use.

But with these dispensers as well, there still is the problem that the mixing ratio of the two components can change depending on the filling level of the formulations in the chambers, and the different flow behavior of the two formulations, and with a decline in the pressure within the dispenser. This poses the great risk that the composition of the application mixture changes during its removal. The associated variations in the color result are very greatly undesired by the consumer.

It has not been possible so far to provide oxidative hair color changing products that are based on a multi-chamber system and enable dosing of the application mixture with a constant, defined composition. The attempt was made in DE 10 2007 056 935 A1 to assure the constancy of the mixing ratio by developing dispensers with a special control mechanism. This mechanism, however, does not rule out incorrect operation on the part of the consumer.

It is therefore desirable to provide a new product for the oxidative coloring and/or lightening of keratinic fibers, which product is based on a dispenser that enables the removal of the finished application mixture. In this case, the application mixture removed via the dispenser should be defined and constant with respect to its composition and not change as a function of the degree of dispenser filling.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A product for the oxidative coloring and/or lightening of keratinic fibers includes (1) a dispenser, which has two chambers (A) and (B) separate from one another, and has an outlet opening (C), which is in communication with chamber (A) and with chamber (B); (2) an aqueous cosmetic preparation (a) in chamber (A) having a viscosity V1 of 1,000 to 100,000 mPas (22° C./Brookfield viscometer/spindle 5/4 rpm); and (3) an aqueous cosmetic preparation (a) in chamber (B) having a viscosity V2 of 1,000 to 100,000 mPas (22° C./Brookfield viscometer/spindle 5/4 rpm). The ratio of the viscosities V1/V2 has a value of 0.3 to 3.0.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it was now found that it is possible to remove an application mixture of invariable composition from a multi-chamber dispenser, if the two preparations in the multi-chamber dispenser are precisely matched with respect to their viscosities.

A first aspect of the invention is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 1000 to 100,000 mPas (22° C./Brookfield viscometer/spindle 5/4 rpm),
(3) an aqueous cosmetic preparation (a) in chamber (B) having a viscosity V2 of 1000 to 100,000 mPas (22° C./Brookfield viscometer/spindle 5/4 rpm),
characterized in that the ratio of the viscosities V1/V2 has a value of 0.3 to 3.0.

Keratinic fibers, keratin-containing fibers, or keratin fibers are to be understood to mean pelts, wool, feathers, and in particular human hair. Although the products of the invention are primarily suitable for lightening and coloring keratin fibers, in principle nothing precludes use in other fields as well.

Preparations (a) and (b) located in chambers (A) and (B) are based in each case on an aqueous cosmetic carrier, for example, on a suitable aqueous, alcoholic, or aqueous-alcoholic carrier. For the purposes of oxidative color changing, such carriers can be, for example, creams, emulsions, gels, or surfactant-containing foaming solutions as well, such as, for example, shampoos, foam aerosols, foam formulations, or other preparations suitable for use on hair. Preparations (a) and/or (b) are especially preferably creams or emulsions.

The feature, essential to the invention, of the product is the precisely matched viscosity of preparations (a) and (b). Viscosity is a measure of the thickness of a fluid. The term fluid includes, for example, solutions, emulsions, gels, or creams. Viscosity within the meaning of the present invention is taken to mean the dynamic viscosity, which is given in the unit mPas. Viscosity is measured hereby preferably with a Brookfield viscometer (22° C./Brookfield viscometer/spindle 5/4 rpm).

Preparation (a), which is located in chamber (A) of the dispenser, has a viscosity of 1000 to 100,000 mPas (22° C./Brookfield viscometer/spindle 5/4 rpm). Preparation (b), which is located in chamber (B) of the dispenser, has a viscosity V2 of 1000 to 100,000 mPas (22° C./Brookfield viscometer/spindle 5/4 rpm). In this respect, a feature, essential to the invention, of the product is that the ratio of the viscosities V1/V2 has a value of 0.3 to 3.0.

The better the viscosities of the two preparations (a) and (b) are matched to one another, the more constant the composition of the mixture of (a) and (b) during removal from outlet opening (C). The removal of defined and constant amounts of formulations (a) and (b) over the entire application period is made possible in this way. Moreover, an application mixture can be removed from the dispenser, which has the same composition in each removal step, regardless of whether the dispenser is still completely filled or is already partially emptied.

It became apparent in this regard that the composition of the application mixture ((a)+(b)) remains especially constant in particular when the viscosities V1 and V2 are set to very special ranges.

In an especially preferred embodiment, a product for the oxidative coloring and/or lightening of keratinic fibers is characterized in that

preparation (a) has a viscosity of 2,000 to 80,000 mPas, preferably of 4,000 to 60,000 mPas, more preferably of 7,000 to 40,000 mPas, even more preferably of 8000 to 35,000 mPas, and particularly preferably of 10,000 to 30,000 mPas (22° C./Brookfield viscometer/spindle 5/4 rpm), and

preparation (b) has a viscosity of 2,000 to 80,000 mPas, preferably of 4,000 to 60,000 mPas, more preferably of 7000 to 40,000 mPas, even more preferably of 8000 to 35,000 mPas, and particularly preferably of 10,000 to 30,000 mPas (22° C./Brookfield viscometer/spindle 5/4 rpm).

Preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 2000 to 80,000 mPas (22° C./Brookfield viscometer/spindle 5/4 rpm),
(3) an aqueous cosmetic preparation (b) in chamber (B) having a viscosity V2 of 2000 to 80,000 mPas (22° C./Brookfield viscometer/spindle 5/4 rpm),
characterized in that the ratio of the viscosities V1/V2 has a value of 0.3 to 3.0.

Preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 4000 to 60,000 mPas (22° C./Brookfield viscometer/spindle 5/4 rpm),
(3) an aqueous cosmetic preparation (a) in chamber (B) having a viscosity V2 of 4000 to 60,000 mPas (22° C./Brookfield viscometer/spindle 5/4 rpm),
characterized in that the ratio of the viscosities V1/V2 has a value of 0.3 to 3.0.

Preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 7,000 to 40,000 mPas (22° C./Brookfield viscometer/spindle 5/4 rpm),
(3) an aqueous cosmetic preparation (a) in chamber (B) having a viscosity V2 of 7,000 to 40,000 mPas (22° C./Brookfield viscometer/spindle 5/4 rpm),
characterized in that the ratio of the viscosities V1/V2 has a value of 0.3 to 3.0.

Very especially preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 8,000 to 35,000 mPas (22° C./Brookfield viscometer/spindle 5/4 rpm),
(3) an aqueous cosmetic preparation (a) in chamber (B) having a viscosity V2 of 8,000 to 35,000 mPas (22° C./Brookfield viscometer/spindle 5/4 rpm),
characterized in that the ratio of the viscosities V1/V2 has a value of 0.3 to 3.0.

Very especially preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 10,000 to 30,000 mPas (22° C./Brookfield viscometer/spindle 5/4 rpm),
(3) an aqueous cosmetic preparation (a) in chamber (B) having a viscosity V2 of 10,000 to 30,000 mPas (22° C./Brookfield viscometer/spindle 5/4 rpm),
characterized in that the ratio of the viscosities V1/V2 has a value of 0.3 to 3.0.

The very best results were obtained when both preparation (a) and preparation (b) were adjusted to a viscosity in the range of 8,000 to 35,000, very especially preferably 10,000 to 30,000 mPas (22° C./Brookfield viscometer/spindle 5/4 rpm). At suitably adjusted viscosities, the application mixture not only had a constant composition, which was independent of dispenser geometry, but an optimal thorough mixing also occurred during removal via the outlet opening, so that the application mixture was also especially homogeneous. The color result was also especially uniform because of this.

In another likewise preferred embodiment, a product for the oxidative coloring and/or lightening of keratinic fibers is characterized in that

preparation (a) has a viscosity of 1000 to 100,000 mPas, preferably of 2000 to 80,000 mPas, more preferably of 4000 to 60,000 mPas, even more preferably of 7000 to 50,000 mPas, and particularly preferably of 10,000 to 45,000 mPas (22° C./Brookfield viscometer/spindle 5/4 rpm), and

preparation (b) has a viscosity of 2000 to 80,000 mPas, preferably of 4000 to 60,000 mPas, more preferably of 7000 to 40,000 mPas, even more preferably of 8000 to 35,000 mPas, and particularly preferably of 10,000 to 30,000 mPas (22° C./Brookfield viscometer/spindle 5/4 rpm).

The better the viscosities of the two preparations (a) and (b) are matched to one another, the more greatly alike as well is the composition of partial amounts of the application mixture, removed from the dispenser at different times of the application process.

In another very especially preferred embodiment, a product for the oxidative coloring and/or lightening of keratinic fibers is characterized in that the ratio of the viscosities V1/V2 has a value of 0.5 to 2.0, preferably of 0.6 to 1.8, more preferably of 0.7 to 1.6, even more preferably of 0.8 to 1.2, and particularly preferably of 0.9 to 1.1.

Preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 2000 to 80,000 mPas (22° C./Brookfield viscometer/spindle 5/4 rpm),
(3) an aqueous cosmetic preparation (b) in chamber (B) having a viscosity V2 of 2000 to 80,000 mPas (22° C./Brookfield viscometer/spindle 5/4 rpm),
characterized in that the ratio of the viscosities V1/V2 has a value of 0.5 to 2.0.
Preferred is a product for the oxidative coloring and/or lightening of keratinic fibers, including
(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 4,000 to 60,000 mPas (22° C./Brookfield viscometer/spindle 5/4 rpm),
(3) an aqueous cosmetic preparation (a) in chamber (B) having a viscosity V2 of 4,000 to 60,000 mPas (22° C./Brookfield viscometer/spindle 5/4 rpm),
characterized in that the ratio of the viscosities V1/V2 has a value of 0.5 to 2.0.

Preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 7,000 to 40,000 mPas (22° C./Brookfield viscometer/spindle 5/4 rpm),
(3) an aqueous cosmetic preparation (a) in chamber (B) having a viscosity V2 of 7,000 to 40,000 mPas (22° C./Brookfield viscometer/spindle 5/4 rpm),
characterized in that the ratio of the viscosities V1/V2 has a value of 0.6 to 1.8.

Very especially preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 8,000 to 35,000 mPas (22° C./Brookfield viscometer/spindle 5/4 rpm),
(3) an aqueous cosmetic preparation (a) in chamber (B) having a viscosity V2 of 8,000 to 35,000 mPas (22° C./Brookfield viscometer/spindle 5/4 rpm),
characterized in that the ratio of the viscosities V1/V2 has a value of 0.7 to 1.6.

Very especially preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 10,000 to 30,000 mPas (22° C./Brookfield viscometer/spindle 5/4 rpm),
(3) an aqueous cosmetic preparation (a) in chamber (B) having a viscosity V2 of 10,000 to 30,000 mPas (22° C./Brookfield viscometer/spindle 5/4 rpm),
characterized in that the ratio of the viscosities V1/V2 has a value of 0.8 to 1.2.

Very especially preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 10,000 to 30,000 mPas (22° C./Brookfield viscometer/spindle 5/4 rpm),
(3) an aqueous cosmetic preparation (a) in chamber (B) having a viscosity V2 of 10,000 to 30,000 mPas (22° C./Brookfield viscometer/spindle 5/4 rpm),
characterized in that the ratio of the viscosities V1/V2 has a value of 0.9 to 1.1.

Very especially preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 10,000 to 30,000 mPas (22° C./Brookfield viscometer/spindle 5/4 rpm),
(3) an aqueous cosmetic preparation (a) in chamber (B) having a viscosity V2 of 10,000 to 45,000 mPas (22° C./Brookfield viscometer/spindle 5/4 rpm),
characterized in that the ratio of the viscosities V1/V2 has a value of 0.7 to 1.6.

The flow behavior of cosmetic formulations can be influenced, for example, by the addition of consistency imparting agents, thickeners, and/or emulsifiers. In the case of the oxidative color-changing agents, the application mixture is prepared from two components, which include different reactive ingredients (preparation (a): oxidation dye precursors, preparation (b): hydrogen peroxide) and which differ greatly both with respect to their further components and also with respect to their consistency. Thus, preparation (a) is usually made with alkaline and has a higher viscosity; preparation (b) includes the oxidizing agent, which is made acidic and in the most cases has a lower viscosity.

It has now become apparent that the rheological properties of the two preparations (a) and (b) despite their different pH values can be matched very well to one another, if both preparations include the same ingredients in high proportions.

In a further, very especially preferred embodiment, a product for the oxidative coloring and/or lightening of keratinic fibers is characterized in that at least 70% by weight, preferably at least 80% by weight, more preferably at least 85% by weight, and very especially preferably at least 90% by weight of the ingredients, included in preparations (a) and (b), are the same.

“Same ingredients” within the meaning of the present invention is understood when preparations (a) and (b) include the same substances or the same raw materials.

Especially preferred, furthermore, is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 8,000 to 35,000 mPas (22° C./Brookfield viscometer/spindle 5/4 rpm),
(3) an aqueous cosmetic preparation (a) in chamber (B) having a viscosity V2 of 8,000 to 35,000 mPas (22° C./Brookfield viscometer/spindle 5/4 rpm),
characterized in that

the ratio of the viscosities V1/V2 has a value of 0.3 to 3.0 and

at least 70% by weight, preferably at least 80% by weight, more preferably at least 85% by weight, and very especially preferably at least 90% by weight of the ingredients, included in preparations (a) and (b), are the same.

The ingredients that are the same in preparations (a) and (b) can be in particular fatty components, thickening polymers, anionic surfactants, cationic surfactants, zwitterionic or amphoteric surfactants, nonionic surfactants, silicones, solvents, and/or water.

It is also of particular advantage in regard to the matching of the rheological properties of preparations (a) and (b), if not only the ingredients in preparations (a) and (b) are the same to a large percentage, but also if both preparations include the essential classes of substances in each case in defined mass ratios to one another.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that
at least one, preferably at least two, and particularly preferably at least three of the weight ratios from the group (G1a)/(G1b), (G2a)/(G2b), (G3a)/(G3b), (G4a)/(G4b) (G5a)/(G5b), and (G6a)/(G6b) have a value of 0.8 to 1.2.

In this case, the total amounts by weight (G1a), (G2a), (G3a), (G4a), (G5a), (G6a), and (G1b) are given in each case as a percentage by weight, whereby the reference basis is the total weight of preparation (a). In this case, the total amounts by weight (G2b), (G3b), (G4b), (G5b), and (G6b) are given in each case as a percentage by weight, whereby the reference basis is the total weight of preparation (b).

“Fatty components” within the meaning of the invention are understood to mean organic compounds with a solubility in water at room temperature (22° C.) and atmospheric pressure (760 mm Hg) of less than 1% by weight, preferably of less than 0.1% by weight.

The definition of fatty components includes explicitly only uncharged (i.e., nonionic) compounds. Fatty components have at least one saturated or unsaturated alkyl group having at least 8 C atoms. The molar weight of the fatty components is a maximum of 5,000 g/mol, preferably a maximum of 2,500 g/mol, and particularly preferably a maximum of 1,000 g/mol. The fatty components are neither polyoxyalkylated nor polyglycerylated compounds.

Preferred fatty components in this regard are understood to mean components from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons. Only nonionic substances are regarded explicitly as fatty components within the meaning of the present invention. Charged compounds such as, for example, fatty acids and salts thereof are not understood to be a fatty component.

C₁₂-C₃₀ fatty alcohols can be saturated, mono- or polyunsaturated, linear or branched fatty alcohols having 12 to 30 C atoms.

Examples of preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol), and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidonyl alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol).

Preferred representatives of branched fatty alcohols are 2-octyldodecanol, 2-hexyldodecanol, and/or 2-butyldodecanol.

A C₁₂-C₃₀ fatty acid triglyceride within the meaning of the present invention is understood to mean the triesters of the trihydric alcohol, glycerol, with three equivalents of fatty acids. In this regard, both structurally similar and also different fatty acids can be involved in ester formations within a triglyceride molecule.

Fatty acids according to the invention are understood to mean saturated or unsaturated, unbranched or branched, unsubstituted or substituted C₁₂-C₃₀ carboxylic acids. Unsaturated fatty acids can be mono- or polyunsaturated. In the case of an unsaturated fatty acid, the C—C double bond(s) thereof can have the cis or trans configuration.

The fatty acid triglycerides are notable for a particular suitability, in which at least one of the ester groups originating from glycerol is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

The fatty acid triglycerides can also be of natural origin. The fatty acid triglycerides, occurring in soybean oil, peanut oil, olive oil, sunflower oil, macadamia nut oil, Moringa oil, apricot kernel oil, Marula oil, and/or optionally hydrogenated castor oil, or mixtures thereof are especially suitable for use in the product of the invention.

A C₁₂-C₃₀ fatty acid monoglyceride is understood to mean the monoester of the trihydric alcohol glycerol with a fatty acid equivalent. In this case, either the middle hydroxy group of glycerol or the terminal hydroxy group of glycerol can be esterified with the fatty acid.

The C₁₂-C₃₀ fatty acid monoglycerides are notable for a particular suitability, in which a hydroxy group of glycerol is esterified with a fatty acid, whereby the fatty acids are selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,14Z)-eicosa-5,8,11,14-tetraenoic acid], and/or nervonic acid [(15Z)-tetracos-15-enoic acid].

A C₁₂-C₃₀ fatty acid diglyceride is understood to mean the diester of the trihydric alcohol, glycerol, with two fatty acid equivalents. In this case, either the middle and a terminal hydroxy group of glycerol can be esterified with two fatty acid equivalents, or however both terminal hydroxy groups of glycerol are esterified with one fatty acid in each case. Glycerol can be esterified hereby both with two structurally similar and with two different fatty acids.

The fatty acid diglycerides are notable for a particular suitability, in which at least one of the ester groups originating from glycerol is formed with a fatty acid, which is selected from dodecanoic acid (lauric acid), tetradecanoic acid (myristic acid), hexadecanoic acid (palmitic acid), tetracosanoic acid (lignoceric acid), octadecanoic acid (stearic acid), eicosanoic acid (arachidic acid), docosanoic acid (behenic acid), petroselinic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z,12Z)-octadeca-9,12-dienoic acid, linolenic acid [(9Z,12Z,15Z)-octadeca-9,12,15-trienoic acid, eleostearic acid [(9Z,11E,13E)-octadeca-9,11,3-trienoic acid], arachidonic acid [(5Z,8Z,11Z,13Z)-eicosa-5,8,11,14-tetraenoic acid], and/or nervonic acid [(15Z)-tetracos-15-enoic acid].

Hydrocarbons are compounds consisting exclusively of carbon and hydrogen atoms and having 8 to 80 C atoms. Preferred in this regard are particularly aliphatic hydrocarbons such as, for example, mineral oils, liquid paraffin oils (e.g., liquid paraffin or light liquid paraffin), isoparaffin oils, semisolid paraffin oils, paraffin waxes, hard paraffin (solid paraffin), Vaseline, and polydecene.

Liquid paraffin oils (liquid paraffin and light liquid paraffin) in particular have proven to be suitable paraffin oils. The hydrocarbon is very especially preferably liquid paraffin, also called white oil. Liquid paraffin is a mixture of purified, saturated, aliphatic hydrocarbons, which consists for the most part of hydrocarbon chains with a C-chain distribution of 25 to 35 C atoms.

Preferred fatty components are selected from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and/or hydrocarbons. C₁₂-C₃₀ fatty alcohols and/or hydrocarbons are preferred fatty components. Very especially preferred fatty components are C₁₂-C₃₀ fatty alcohols.

The fatty components are included in a total amount by weight (G1a) in preparation (a) (data given in percentages by weight). The calculation basis for the total amount by weight (G1a) is the total weight of preparation (a).

The fatty components are included in a total amount by weight (G1b) in preparation (b) (data given in percentages by weight). The calculation basis for the total amount by weight (G1a) is the total weight of preparation (b).

The fatty components can be used in total amounts by weight (G1a) or (G2a) of 0.1 to 40% by weight, in each case based on the total weight of preparations (a) and (b).

“Thickening polymers” within the meaning of the present invention are understood to mean organic polymeric compounds that increase the viscosity of the water, if they are added in an amount of 0.1% by weight to water (at 22° C. and a pressure of 760 mm Hg). The measurement of the viscosity occurs in this case at 22° C. (22° C./Brookfield viscometer/spindle 1/10 rpm).

Polymeric compounds within the meaning of the present invention are understood to mean compounds, based on repeat units, with a molar weight of more than 5,000 g/mol, preferably of more than 10,000 g/mol, and particularly preferably of more than 20,000 g/mol. The maximum molar weight of the polymers depends on the synthesis conditions, batch size, and the degree of polymerization and preferably does not exceed 10⁸ g/mol.

Compounds based on repeat units have at least 50 repeat units, preferably at least 100 repeat units, and particularly preferably at least 500 repeat units.

The thickening polymers are included in a total amount by weight (G2a) in preparation (a) (data given in percentages by weight). The calculation basis for the total amount by weight (G2a) is the total weight of preparation (a).

The thickening polymers are included in a total amount by weight (G2b) in preparation (b) (data given in percentages by weight). The calculation basis for the total amount by weight (G2b) is the total weight of preparation (b).

The thickening polymers can be cationic, nonionic, and/or anionic polymers.

If one or more compounds from the group of polysaccharides, acrylic acid polymers, acrylic acid copolymers, methacrylic acid polymers, and/or methacrylic acid copolymers are used as thickening polymers, then this is especially preferred.

The term polysaccharides is the collective term for macromolecular carbohydrates whose molecules consist of a large number of glycosidically linked monosaccharide molecules (with a molar mass of at least 5000 g/mol). These carbohydrates can also be chemically modified.

The xanthan gums, alginates, carboxyalkylcelluloses, and hyaluronic acids have a very particular suitability within the group of the polysaccharides.

Xanthan gum is a polysaccharide, which is made up of, inter alia, the structural components: D-glucose, D-mannose, D-glucuronic acid, acetate, and pyruvate, and is also known under the INCI name Xanthan Gum. Xanthan gum carries carboxy groups and is anionic or anionizable. The physiologically acceptable salts of xanthan gum are also novel.

Salts of alginic acid are called alginates (INCI name Algin). Alginates are acidic, carboxy group-containing polysaccharides, consisting of D-mannuronic acid and D-guluronic acid in different ratios, which are linked by 1-4-glycosidic bonds. The alkaline and alkaline earth salts of alginic acids in particular are novel. The use of alginic acid, sodium alginate, potassium alginate, ammonium alginate, and/or calcium alginate has proven especially advantageous in the preparations of the invention.

Carboxyalkylcelluloses are cellulose ethers in which the hydrogen atoms of the hydroxy groups of cellulose are partially or completely substituted by carboxyalkyl groups. A preferred carboxyalkylcellulose is carboxymethylcellulose, which preferably can be used as an anionic polymer in the form of its sodium salt (sodium carboxymethylcellulose).

The basic structural unit of hyaluronic acid (INCI names: Hyaluronic acid, Sodium Hyaluronate) is an amino dissacharide which is made up of D-glucuronic acid and N-acetylglucosamine in a 1-3-glycosidic bond and is connected to the next unit with a β-1-4-glycosidic bond. The sodium and potassium salts of hyaluronic acid have also proven especially suitable within the scope of work leading to this invention.

Preparations (a) and/or (b) can include the thickening polymers in total amounts of weight (G2a) or (G2b) of 0.1 to 20% by weight.

Preparation (a) and/or preparation (b) can also include one or more cationic surfactants. Preparation (a) includes the cationic surfactants in a total amount by weight (G3a) (data given in percentages by weight). The calculation basis for the total amount by weight (G3a) is the total weight of preparation (a).

Preparation (b) includes the cationic surfactants in a total amount by weight (G3b) (data given in percentages by weight). The calculation basis for the total amount by weight (G3b) is the total weight of preparation (b).

Cationic surfactants are generally derived from ammonium ions and have a structure (NR¹R²R³R⁴)⁺ with a correspondingly negatively charged counterion. Other cationic surfactants are, for example, esterquats or imidazolium compounds. Cationic surfactants (Tkat) of the quaternary ammonium compound type, esterquat type, imidazoline type, and amidoamine type can be used particularly preferably according to the invention. Preferred quaternary ammonium compounds are ammonium halides, in particular chlorides and bromides, such as alkyltrimethylammonium chlorides, dialkyldimethylammonium chlorides, and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, and the imidazolium compounds known under the INCI names Quaternium-27 and Quaternium-83. The long alkyl chains of the aforementioned surfactants preferably have 8 to 30 carbon atoms. Typical examples of cationic surfactants are quaternary ammonium compounds and esterquats, particularly quaternized fatty acid trialkanolamine ester salts.

Cationic compounds with behenyl groups, particularly the substances known under the name behentrimonium chloride or bromide (docosanyltrimethylammonium chloride or bromide) can be used particularly preferably according to the invention. Other preferred quaternary ammonium compounds have at least two behenyl groups. These substances are available commercially, for example, under the names Genamin® KDMP (Clariant).

Esterquats are substances, which include both at least one ester function and at least one quaternary ammonium group as a structural element. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Stepantex®, Dehyquart®, and Armocare®. The products Armocare® VGH-70, an N,N-bis(2-palmitoyloxyethyl)dimethylammonium chloride, and Dehyquart® F-75, Dehyquart® C-4046, Dehyquart® L80, and Dehyquart® AU-35 are examples of such esterquats.

Preparations (a) and/or (b) can include as other cationic surfactants at least one quaternary imidazoline compound, i.e., a compound that has a positively charged imidazoline ring. The formula (Tkat-1) illustrated below shows the structure of these compounds.

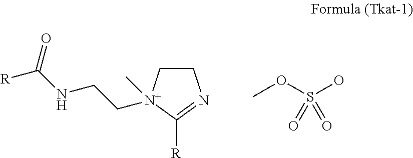

The groups R independently of one another in each case stand for a saturated or unsaturated, linear or branched hydrocarbon group with a chain length of 8 to 30 carbon atoms. The preferred compounds of the formula (E5-V) include in each case the same hydrocarbon group for R. The chain length of the R groups is preferably 12 carbon atoms. Especially preferred are compounds with a chain length of at least 16 carbon atoms and very especially preferred with at least 20 carbon atoms. A very especially preferred compound of formula I has a chain length of 21 carbon atoms. A commercial product of this chain length is known, for example, under the name Quaternium-91. Methosulfate is shown as the counterion in the formula (Tkat-1). According to the invention, however, halides such as chloride, fluoride, bromide, or phosphates as well are included as counterions.

The compositions of the invention include imidazolines of formula (Tkat-1) in amounts of 0.01 to 20% by weight, preferably in amounts of 0.05 to 10% by weight, and very especially preferably in amounts of 0.1 to 7.5% by weight. The very best results in this case are obtained with amounts of 0.1 to 5% by weight, in each case based on the total composition of the particular agent.

The alkylamidoamines are usually prepared by amidation of natural or synthetic fatty acids and fatty acid cuts with dialkylaminoamines. Stearamidopropyl dimethylamine, commercially available under the name Tegoamid® S 18, represents a compound, especially suitable according to the invention, from this substance group. Alkylamidoamines can be present both as such and converted by protonation in a suitably acidic solution into a quaternary compound in the composition, but they can also be used, of course, as a permanent quaternary compound in the compositions of the invention. Examples of permanently quaternized amidoamines are, for example, the raw materials with the trade name Rewoquat® UTM 50, Lanoquat® DES-50, or Empigen CSC.

A further example of a quaternary sugar derivative that can be used as a cationic surfactant is the commercial product Glucquat®100, a “Lauryl Methyl Gluceth-10 Hydroxypropyl Dimonium Chloride” according to INCI nomenclature.

Finally, cationic compounds with the following general structure are also understood to be cationic surfactants in the present invention:

RCO—X—N⁺R¹R²R³R⁴ A⁻ (Tkat-2)

R herein stands for a substituted or unsubstituted, branched or straight-chain alkyl or alkenyl group having 11 to 35 carbon atoms in the chain,
X stands for —O— or —NR⁵—,
R¹ stands for an alkylene group having 2 to 6 C atoms, which may be unsubstituted or substituted, whereby in the case of a substitution, substitution with an —OH or —NH group is preferred,
R², R³, and R⁴ each independently of one another stand for an alkyl or hydroxyalkyl group having 1 up to 6 C atoms in the chain, whereby the chain can be straight or branched. Examples of groups according to the invention are methyl, ethyl, propyl, isopropyl, butyl, isobutyl, tert-butyl, pentyl, isopentyl, neopentyl, hexyl, isohexyl, hydroxyalkyl, dihydroxyalkyl, hydroxyethyl, hydroxypropyl, dihydroxypropyl, hydroxybutyl, dihydroxybutyl, trihydroxybutyl, trihydroxypropyl, and dihydroxyethyl,
R⁵ stands for hydrogen or a C1 to C6 straight-chain or branched, alkyl or alkenyl group, which can also be substituted by a hydroxy group, in particular methyl, ethyl, propyl, isopropyl, butyl, isobutyl, tert-butyl, pentyl, isopentyl, neopentyl, hexyl, isohexyl, hydroxyethyl, hydroxypropyl, dihydroxypropyl, hydroxybutyl, dihydroxybutyl, trihydroxybutyl, trihydroxypropyl, dihydroxyethyl, and
A- stands for a halide, such as fluoride, chloride, or bromide, an alkyl sulfate, such as methosulfate or ethosulfate, a phosphate, a citrate, tartrate, maleate, or fumarate.

Compounds of one of the following structures are used with preference within this structure class:

CH₃(CH₂)₂₀CONH(CH₂)₃—N⁺(CH₃)₂—CH₂CH₃ A⁻ (Tkat-3)

CH₃(CH₂)₂₀CONH(CH₂)₃—N⁺(CH₃)₂—CH₂(CHOH)CH₂OH A⁻ (Tkat-4)

CH₃(CH₂)₂₀COOCH₂CHOHCH₂—N⁺(CH₃)₃ A⁻ (Tkat-5)

CH₃(CH₂)₂₀CONH(CH₂)₃—N⁺(CH₃)₂—CH₂CH₂OH A⁻ (Tkat-6)

Examples of such commercial products are Schercoquat BAS, Lexquat AMG-BEO, Akypoquat 131, or Incroquat Behenyl HE.

Preparations (a) and/or (b) can include the cationic surfactants in each case in total amounts by weight (G3a) or (G3b) of 0.1 to 40% by weight.

Preparation (a) and/or preparation (b) can also include one or more anionic surfactants. Preparation (a) includes the anionic surfactants in a total amount by weight (G4a) (data given in percentages by weight). The calculation basis for the total amount by weight (G4a) is the total weight of preparation (a). Preparation (b) includes the anionic surfactants in a total amount by weight (G4b) (data given in percentages by weight). The calculation basis for the total amount by weight (G4b) is the total weight of preparation (b).

All anionic surface-active substances, suitable for use on the human body, are suitable as anionic surfactants (Tanion) in the preparations of the invention. These are characterized by an anionic group imparting water solubility, such as, e.g., a carboxylate, sulfate, sulfonate, or phosphate group, and a lipophilic alkyl group having, for instance, 8 to 30 C atoms. In addition, glycol ether or polyglycol ether groups, ester, ether, and amide groups, and hydroxyl groups can be included in the molecule. Typical examples of anionic surfactants are alkylbenzene sulfonates, alkane sulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, acyl lactylates, acyl tartrates, acyl glutamates, acyl aspartates, alkyl oligoglucoside sulfates, protein fatty acid condensates (particularly plant products with a wheat base), and alkyl (ether) phosphates. Provided the anionic surfactants include polyglycol ether chains, these can have a conventional but preferably narrow homolog distribution. Examples of especially suitable anionic surfactants are, each in the form of the sodium, potassium, and ammonium and mono-, di-, and trialkanolammonium salts having 2 to 4 C atoms in the alkanol group,
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or is 1 to 16,
acyl sarcosides having 8 to 24 C atoms in the acyl group,
acyl taurides having 8 to 24 C atoms in the acyl group,
acyl isethionates, having 8 to 24 C atoms in the acyl group, are long-known, skin-friendly surface-active substances, which can be obtained by esterification of fatty acids with the sodium salt of 2-hydroxyethanesulfonic acids (isethionic acid). If fatty acids having 8 to 24 C atoms, therefore, e.g., lauric, myristic, palmitic, or stearic acid or technical fatty acid fractions as well, e.g., the C₁₂-C₁₈ fatty acid fraction, obtainable from coconut fatty acid are used for this esterification, the C₁₂-C₁₈ acyl isethionates preferably suitable according to the invention are obtained. It is known that sodium salts of C₁₂-C₁₈ acyl isethionates like soaps with a fatty acid basis may be brought into a suitable form for transport and application by kneading, milling, extrusion molding, extrusion, cutting, and barring. Needles, granules, noodles, or bars can be produced in this way. Toilet soap pieces and syndets are an application of acyl isethionates.
sulfosuccinic acid mono- and dialkyl esters having 8 to 24 C atoms in the alkyl group, and sulfosuccinic acid monoalkylpolyoxyethyl esters having 8 to 24 C atoms in the alkyl group and 1 to 6 oxyethyl groups. The sulfosuccinic acid monoalkyl (C₈-C₂₄) ester disodium salts are produced by known methods, e.g., in that maleic anhydride is reacted with a fatty alcohol having 8 to 24 C atoms to form the maleic acid monoester of the fatty alcohol and this is sulfated with sodium sulfite to form the sulfosuccinic acid ester. Especially suitable sulfosuccinic acid esters are derived from fatty alcohol fractions having 12 to 18 C atoms, as they can be obtained, e.g., from coconut fatty acid or coconut fatty acid methyl esters by hydrogenation,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or is 1 to 12,
hydroxysulfonates substantially corresponding to at least one of the two following formulas or mixtures and salts thereof, CH₃—(CH₂)_{y}—CHOH—(CH₂)ₚ—(CH—SO₃M)-(CH₂)_{z}—CH₂—O—(CₙH₂ₙO)ₓ—H, and/or CH₃—(CH₂)_{y}—(CH—SO₃M)-(CH₂)ₚ—CHOH—(CH₂)_{z}—CH₂—O—(CₙH₂ₙO)ₓ—H, whereby in both formulas y and z=0 or whole numbers from 1 to 18, p=0, 1, or 2 and the sum (y+z+p) is a number from 12 to 18, x=0 or is a number from 1 to 30 and n is a whole number from 2 to 4 and M=H or alkali, particularly sodium, potassium, lithium, alkaline earth, particularly magnesium, calcium, zinc, and/or an ammonium ion, which optionally may be substituted, particularly mono-, di-, tri-, or tetraammonium ions having C1 to C4 alkyl, alkenyl, or aryl groups,
sulfated hydroxyalkyl polyethylene and/or hydroxyalkylene propylene glycol ethers, of the formula R¹—(CHOSO₃M)-CHR³—(OCHR⁴—CH₂)ₙ—OR² in which R¹ stands for a linear alkyl group having 1 to 24 C atoms, R² for a linear or branched, saturated alkyl group having 1 to 24 C atoms, R³ for hydrogen or a linear alkyl group having 1 to 24 C atoms, R⁴ for hydrogen or a methyl group, and M for hydrogen, ammonium, alkylammonium, alkanolammonium, where the alkyl and alkanol groups each have 1 to 4 C atoms, or a metal atom selected from lithium, sodium, potassium, calcium, or magnesium, and n for a number in the range of 0 to 12, and furthermore the total number of C atoms in R¹ and R³ is 2 to 44,
sulfonates of unsaturated fatty acids having 8 to 24 C atoms and 1 to 6 double bonds,
esters of tartaric acid and citric acid with alcohols, representing adducts of, for instance, 2 to 15 molecules of ethylene oxide and/or propylene oxide to fatty alcohols having 8 to 22 C atoms,
alkyl and/or alkenyl ether phosphates of the formula, R¹(OCH₂CH₂)ₙ—O—(PO—OX)—OR², in which R¹ preferably stands for an aliphatic hydrocarbon group having 8 to 30 carbon atoms, R² for hydrogen, a group (CH₂CH₂O)ₙR², or X, n for numbers from 1 to 10, and X for hydrogen, an alkali metal or alkaline earth metal or NR³R⁴R⁵R⁶, where R³ to R⁶ independently of one another stand for hydrogen or a C₁ to C₄ hydrocarbon group,
sulfated fatty acid alkylene glycol esters of the formula RCO(AlkO)ₙSO₃M in which RCO— stands for a linear or branched, aliphatic, saturated and/or unsaturated acyl group having 6 to 22 C atoms, Alk for CH₂CH₂, CHCH₃CH₂, and/or CH₂CHCH₃, n for numbers from 0.5 to 5, and M for a metal, such as an alkali metal, particularly sodium, potassium, lithium, alkaline earth metal, particularly magnesium, calcium, zinc, or ammonium ion, such as ⁺NR³R⁴R⁵R⁶, whereby R³ to R⁶ independently of one another stand for hydrogen or a C1 to C4 hydrocarbon group,
monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)_{z}—SO₃X,
in which R⁸CO stands for a linear or branched acyl group having 6 to 22 carbon atoms, x, y, and z in total for 0 or for numbers from 1 to 30, preferably 2 to 10, and X for an alkali or alkaline earth metal. Typical examples within the meaning of the invention for suitable monoglyceride (ether) sulfates are the reaction products of lauric acid monoglyceride, coconut fatty acid monoglyceride, palmitic acid monoglyceride, stearic acid monoglyceride, oleic acid monoglyceride, and tallow fatty acid monoglyceride, as well as the ethylene oxide adducts thereof with sulfur trioxide or chlorosulfonic acid in the form of their sodium salts. Preferably, monoglyceride sulfates are used in which R⁸CO stands for a linear acyl group having 8 to 18 carbon atoms,
amide ether carboxylic acids, R¹—CO—NR²—CH₂CH²⁻O—(CH₂C₂CH₂O)ₙCH₂COOM, with R¹ as a straight-chain or branched alkyl or alkenyl group with a number of carbon atoms in the chain of 2 to 30, n stands for a whole number from 1 to 20, and R² stands for hydrogen, a methyl, ethyl, propyl, isopropyl, n-butyl, t-butyl, or isobutyl group, and M stands for hydrogen or a metal such as an alkali metal, particularly sodium, potassium, lithium, alkaline earth metal, particularly magnesium, calcium, zinc, or an ammonium ion, such as ⁺NR³R⁴R⁵R⁶, whereby R³ to R⁶ independently of one another stand for hydrogen or a C1 to C4 hydrocarbon group. Such products can be obtained, for example, from the company Chem-Y under the product name Akypo®.
acyl glutamates of the formula XOOC—CH₂CH₂CH(C(NH)OR)—COOX, in which RCO stands for a linear or branched acyl group having 6 to 22 carbon atoms and 0 and/or 1, 2, or 3 double bonds and X for hydrogen, an alkali and/or alkaline earth metal, ammonium, alkyl ammonium, alkanol ammonium, or glucammonium,
condensation products of a water-soluble salt of a water-soluble protein hydrolysate-fatty acid condensation product. These are prepared by condensation of C8-C30 fatty acids, preferably of fatty acids having 12 to 18 C atoms, with amino acids, mono-, di-, and water-soluble oligopeptides, and mixture of such products, as they accumulate in the hydrolysis of proteins. These protein hydrolysate-fatty acid condensation products are neutralized with a base and are then present preferably as alkali, ammonium, mono-, di-, or trialkanol ammonium salt. Such products have been commercially available for a long time under the trademarks Lamepon®, Maypon®, Gluadin®, Hostapon® KCG, or Amisoft®.

Preferred anionic surfactants are alkyl sulfates, alkyl polyglycol ether sulfates, and ether carboxylic acids having 10 to 18 C atoms in the alkyl group and up to 12 glycol ether groups in the molecule, sulfosuccinic acid mono- and dialkyl esters having 8 to 24 C atoms in the alkyl group, and sulfosuccinic acid monoalkyl polyoxyethyl esters having 8 to 24 C atoms in the alkyl group and 1 to 6 oxyethyl groups, monoglyceride sulfates, alkyl and alkenyl ether phosphates, and protein fatty acid condensates.

Preparations (a) and/or (b) can include the anionic surfactants in each case in total amounts by weight (G4a) or (G4b) of 0.1 to 40% by weight.

Preparation (a) and/or preparation (b) can also include one or more nonionic surfactants. Preparation (a) includes the nonionic surfactants in a total amount by weight (G5a) (data given in percentages by weight). The calculation basis for the total amount by weight (G5a) is the total weight of preparation (a). Preparation (b) includes the anionic surfactants in a total amount by weight (G5b) (data given in percentages by weight). The calculation basis for the total amount by weight (G5b) is the total weight of preparation (b).

Nonionic surfactants include as the hydrophilic group, e.g., a polyol group, a polyalkylene glycol ether group, or a combination of polyol and polyglycol ether groups. Such compounds are, for example,
adducts of 2 to 50 mol of ethylene oxide and/or 0 to 5 mol of propylene oxide to linear and branched fatty alcohols having 6 to 30 C atoms, fatty alcohol polyglycol ethers or fatty alcohol polypropylene glycol ethers or mixed fatty alcohol polyethers,
adducts of 2 to 50 mol of ethylene oxide and/or 0 to 5 mol of propylene oxide to linear and branched fatty acids having 6 to 30 C atoms, fatty acid polyglycol ethers or fatty acid polypropylene glycol ethers or mixed fatty acid polyethers,
adducts of 2 to 50 mol of ethylene oxide and/or 0 to 5 mol of propylene oxide to linear and branched alkylphenols having 8 to 15 C atoms in the alkyl group, alkyl phenol polyglycol ethers or alkyl polypropylene glycol ethers, or mixed alkyl phenol polyethers,
adducts, end-capped with a methyl or C₂-C₆ alkyl group, of 2 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group, such as, for example, the types obtainable under the marketing names Dehydol® LS and Dehydol® LT (Cognis),
C₁₂-C₃₀ fatty acid monoesters and diesters of adducts of 1 to 30 mol of ethylene oxide to glycerol,
adducts of 5 to 60 mol of ethylene oxide to castor oil and hydrogenated castor oil,
polyol fatty acid esters, such as, for example, the commercial product Hydagen® HSP (Cognis) or Sovermol® types (Cognis),
alkoxylated triglycerides,
alkoxylated fatty acid alkyl esters of the formula (Tnio-1)

R¹CO—(OCH₂CHR²)_{w}OR³ (Tnio-1)
in which R¹CO stands for a linear or branched, saturated and/or unsaturated acyl group having 6 to 22 carbon atoms, R² for hydrogen or methyl, R³ for linear or branched alkyl groups having 1 to 4 carbon atoms, and w for a number from 1 to 20,
amine oxides,
hydroxy mixed ethers, as they are described, for example, in DE-OS 19738866,
sorbitan fatty acid esters and adducts of ethylene oxide to sorbitan fatty acid esters, such as, for example, polysorbates,
sugar fatty acid esters and adducts of ethylene oxide to sugar fatty acid esters,
adducts of ethylene oxide to fatty acid alkanolamides and fatty amines,
sugar surfactants of the alkyl and alkenyl oligoglycoside type according to formula (E4-II),

R⁴O-[G]ₚ (Tnio-2)
in which R⁴ stands for an alkyl or alkenyl group having 4 to 22 carbon atoms, G for a sugar group having 5 or 6 carbon atoms, and p for numbers from 1 to 10. The alkyl and alkenyl oligoglycosides can be derived from aldoses or ketoses having 5 or 6 carbon atoms, preferably from glucose. The preferred alkyl and/or alkenyl oligoglycosides therefore are alkyl and/or alkenyl oligoglucosides. The subscript p in the general formula (Tnio-2) indicates the degree of oligomerization (DP), i.e., the distribution of mono- and oligoglycosides and stands for a number between 1 and 10. Whereas in individual molecules p must always be an integer and here can assume primarily the values p=1 to 6, the value of p for a specific alkyl oligoglycoside is an analytically determined mathematical quantity, which is usually a fraction. Preferably alkyl and/or alkenyl oligoglycosides with an average degree of oligomerization p of 1.1 to 3.0 are used. Alkyl and/or alkenyl oligoglycosides whose degree of oligomerization is less than 1.7 and particularly between 1.2 and 1.4 are preferred from an application technology standpoint. The alkyl or alkenyl group R⁴ can be derived from primary alcohols having 4 to 11, preferably 8 to 10 carbon atoms. Typical examples are butanol, hexyl alcohol, caprylic alcohol, capric alcohol, and undecyl alcohol and technical mixtures thereof, as are obtained, for example, in the hydrogenation of technical fatty acid methyl esters or during the hydrogenation of aldehydes from Roelen's oxo synthesis. Alkyl oligoglucosides with a chain length of C₈-C₁₀ (DP=1 to 3) are preferred, which accumulate as forerun during the distillative separation of technical C₈-C₁₈ coconut fatty alcohol and can be contaminated with a proportion of less than 6% by weight of C₁₂ alcohol, as well as alkyl oligoglucosides based on technical C_{9/11} oxo alcohols (DP=1 to 3). The alkyl or alkenyl group R¹⁵ can be derived further also from primary alcohols having 12 to 22, preferably 12 to 14 carbon atoms. Typical examples are lauryl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical mixtures thereof, which can be obtained as described above. Alkyl oligoglucosides based on hydrogenated C_{12/14} coconut alcohol with a DP of 1 to 3 are preferred.
sugar surfactants of the fatty acid N-alkylpolyhydroxyalkylamide type, a nonionic surfactant of the formula (Tnio-3),

R⁵CO—NR⁶—[Z] (Tnio-3)
in which R⁵CO stands for an aliphatic acyl group having 6 to 22 carbon atoms, R⁶ for hydrogen, an alkyl or hydroxyalkyl group having 1 to 4 carbon atoms, and [Z] for a linear or branched polyhydroxyalkyl group having 3 to 12 carbon atoms and 3 to 10 hydroxyl groups. Fatty acid N-alkylpolyhydroxyalkylamides are known substances, which typically can be obtained by reductive amination of a reducing sugar with ammonia, an alkylamine or an alkanolamine and subsequent acylation with a fatty acid, a fatty acid alkyl ester, or a fatty acid chloride. Preferably, the fatty acid N-alkylpolyhydroxyalkylamides are derived from reducing sugars having 5 or 6 carbon atoms, in particular from glucose. The preferred fatty acid N-alkylpolyhydroxyalkylamides are therefore fatty acid N-alkylglucamides, as they are given by the formula (Tnio-4):

R⁷CO—(NR⁸)—CH₂—[CH(OH)]₄—CH₂OH (Tnio-4).
Preferably, used as fatty acid N-alkylpolyhydroxyalkylamides are glucamides of the formula (Tnio-4) in which R⁸ stands for hydrogen or an alkyl group and R⁷CO stands for the acyl group of caproic acid, caprylic acid, capric acid, lauric acid, myristic acid, palmitic acid, palmitoleic acid, stearic acid, isostearic acid, oleic acid, elaidic acid, petroselic acid, linoleic acid, linolenic acid, arachic acid, gadoleic acid, behenic acid, or erucic acid, or technical mixtures thereof. Particularly preferred are fatty acid N-alkylglucamides of the formula (Tnio-4) which are obtained by reductive amination of glucose with methylamine and subsequent acylation with lauric acid or C_{12/14} coconut fatty acid or a corresponding derivative. Furthermore, the polyhydroxyalkylamides can also be derived from maltose and palatinose.

Other typical examples of nonionic surfactants are fatty acid amide polyglycol ethers, fatty amine polyglycol ethers, mixed ethers, or mixed formals, protein hydrolysates (particularly plant products on a wheat base), and polysorbates.

Alkylene oxide adducts to saturated linear fatty alcohols and fatty acids each having 2 to 30 mol of ethylene oxide per mole of fatty alcohol or fatty acid, as well as sugar surfactants, have proven to be preferred nonionic surfactants.

Preparations (a) and/or (b) can include the nonionic surfactants in each case in total amounts by weight (G5a) or (G5b) of 0.1 to 40% by weight.

Preparation (a) and/or preparation (b) can also include one or more zwitterionic surfactants. Preparation (a) includes the zwitterionic surfactants in a total amount by weight (G6a) (data given in percentages by weight). The calculation basis for the total amount by weight (G6a) is the total weight of preparation (a). Preparation (b) includes the zwitterionic surfactants in a total amount by weight (G6b) (data given in percentages by weight). The calculation basis for the total amount by weight (G6b) is the total weight of preparation (b).

Surface-active compounds that have at least one quaternary ammonium group and at least one —COO⁽⁻⁾ or —SO₃⁽⁻⁾ group in the molecule are called zwitterionic surfactants. Especially suitable zwitterionic surfactants are the so-called betaines, such as the N-alkyl-N,N-dimethylammonium glycinates, for example, coco alkyl dimethylammonium glycinate and N-acyl-aminopropyl-N,N-dimethylammonium glycinates, for example, cocoacylaminopropyldimethylammonium glycinate, and 2-alkyl-3-carboxymethyl-3-hydroxyethyl imidazolines each having 8 to 18 C atoms in the alkyl or acyl group, as well as cocoacylaminoethyl hydroxyethyl carboxymethyl glycinate. A preferred zwitterionic surfactant is the fatty acid amide derivative known under the INCI name of Cocamidopropyl Betaine.

As previously described, preparations (a) and/or (b) in another very especially preferred embodiment include

one or more fatty components in a total amount by weight (G1a) or (G1b)

one or more thickening polymers in a total amount by weight (G2a) and/or (G2b)

one or more cationic surfactants in a total amount by weight (G3a) and/or (G3b)

one or more anionic surfactants in a total amount by weight (G4a) and/or (G4b)

one or more nonionic surfactants in a total amount by weight (G5a) and/or (G5b)

one or more zwitterionic surfactants in a total amount by weight (G6a) or (G6b)

The feature, essential to the invention, of this especially preferred embodiment consists hereby in that at least one, preferably at least two, and particularly preferably at least three of the weight ratios from the group (G1a)/(G1b), (G2a)/(G2b), (G3a)/(G3b), (G4a)/(G4b) (G5a)/(G5b), and (G6a)/(G6b) have a value of 0.8 to 1.2.

In principle, one or more total amounts by weight from the group (G1a), (G2a), (G3a), (G4a), (G5a), and (G6a) can also be 0. Likewise, one or more total amounts by weight from the group (G1b), (G2b), (G3b), (G4b), (G5b), and (G6b) can also be 0. In this case, however, the requirement must be fulfilled that at least one (preferably at least two, in particular at least preferably three) weight ratio(s) from the group (G1a)/(G1b), (G2a)/(G2b), (G3a)/(G3b), (G4a)/(G4b) (G5a)/(G5b), and (G6a)/(G6b) has (have) a value of 0.8 to 1.2. This means that at least one (preferably at least two, especially preferably at least three) of the amounts of (G1a), (G2a), (G3a), (G4a), (G5a), and (G6a) must be different from 0 and, furthermore, at least one (preferably at least two, especially preferably at least three) of the amounts of (G1b), (G2b), (G3b), (G4b), (G5b), and (G6b) must be dissimilar.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2

In this case, the fatty alcohols are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G2a)/(G2b) has a value of 0.8 to 1.2.

In this case, the fatty alcohols and the thickening polymers are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G3a)/(G3b) has a value of 0.8 to 1.2.

In this case, the fatty alcohols and the cationic surfactants are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G4a)/(G4b) has a value of 0.8 to 1.2.

In this case, the fatty alcohols and the anionic surfactants are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G5a)/(G5b) has a value of 0.8 to 1.2.

In this case, the fatty alcohols and the nonionic surfactants are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G6a)/(G6b) has a value of 0.8 to 1.2.

In this case, the fatty alcohols and the zwitterionic surfactants are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G2a)/(G2b) has a value of 0.8 to 1.2, and

the weight ratio (G3a)/(G3b) has a value of 0.8 to 1.2

In this case, the fatty alcohols, the thickening polymers, and the cationic surfactants are mandatory components in preparations (a) and (b); the other ingredients are optional.

Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G2a)/(G2b) has a value of 0.8 to 1.2, and

the weight ratio (G4a)/(G4b) has a value of 0.8 to 1.2

In this case, the fatty alcohols, the thickening polymers, and the anionic surfactants are mandatory components in preparations (a) and (b); the other ingredients are optional. Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b),

characterized in that

the weight ratio (G1a)/(G1b) has a value of 0.8 to 1.2, and

the weight ratio (G2a)/(G2b) has a value of 0.8 to 1.2, and

the weight ratio (G5a)/(G5b) has a value of 0.8 to 1.2

In this case, the fatty alcohols, the thickening polymers, and the nonionic surfactants are mandatory components in preparations (a) and (b); the other ingredients are optional. Also very especially preferred is a product for the oxidative coloring and/or lightening of keratinic fibers, whereby preparation (a) includes, in each case based on its total weight,

one or more fatty components in a total amount by weight (G1a) and/or

one or more thickening polymers in a total amount by weight (G2a) and/or

one or more cationic surfactants in a total amount by weight (G3a) and/or

one or more anionic surfactants in a total amount by weight (G4a) and/or

one or more nonionic surfactants in a total amount by weight (G5a) and/or

one or more zwitterionic surfactants in a total amount by weight (G6a) and/or,

whereby preparation (b) includes, in each case based on its total weight,

one or more fatty components in a total amount by weight (G1b) and/or

one or more thickening polymers in a total amount by weight (G2b) and/or

one or more cationic surfactants in a total amount by weight (G3b) and/or

one or more anionic surfactants in a total amount by weight (G4b) and/or

one or more nonionic surfactants in a total amount by weight (G5b) and/or

one or more zwitterionic surfactants in a total amount by weight (G6b)

characterized in that

the weight ratio (G1a)/(G1b) has a value of 0.8 to 1.2, and

the weight ratio (G2a)/(G2b) has a value of 0.8 to 1.2, and

the weight ratio (G6a)/(G6b) has a value of 0.8 to 1.2.

In this case, the fatty alcohols, the thickening polymers, and the zwitterionic surfactants are mandatory components in preparations (a) and (b); the other ingredients are optional.

Very good results in regard to the solution to the problem posed according to the invention were also obtained, if the aforementioned ingredients (G1) to (G6) in preparations (a) and (b) were used in such total amounts that the sum of the total weight of the ingredients (G1) to (G6) in both formulations was also the same in each case, i.e., if the weight ratio

[(G1a)+(G2a)+(G3a)+(G4a)+(G5a)+(G6a)]/[(G1b)+(G2b)+(G3b)+(G4b)+(G5b)+(G6b)]

was also within a value range of 0.6 to 1.4. The closer this weight ratio as well approached a value of 1, the more homogeneous were the application mixtures obtained during use of the product of the invention and the more uniform as well was the color result obtained in the dyeing.

In a further especially preferred embodiment, a product for the oxidative coloring and/or lightening of keratinic fibers is characterized for this reason in that the ratio of the sum of the total amounts by weight G1a, G2a, G3a, G4a, G5a, and G6a in preparation (a) to the sum of the total amounts by weight G1b, G2b, G3b, G4b, G5b, and G6b in preparation (b), i.e., the ratio

[(G1a)+(G2a)+(G3a)+(G4a)+(G5a)+(G6a)]/[(G1b)+(G2b)+(G3b)+(G4b)+(G5b)+(G6b)]

has a value of 0.6 to 1.4, preferably 0.7 to 1.3, more preferably 0.8 to 1.2, and particularly preferably 0.9 to 1.1.

If the total amounts of very special ingredients in preparations (a) and (b) are the same, thus this is especially effective in matching the flow behavior of both preparations. In this regard, matching of the total amounts of fatty components and anionic surfactants has proven especially advantageous.

Also especially preferred is a product for the oxidative coloring and/or lightening of keratinic fibers, whereby preparation (a) includes, based on its total weight,

one or more fatty components in a total amount by weight (G1a) and

one or more anionic surfactants in a total amount by weight (G4a), whereby preparation (b) includes, based on its total weight,

one or more fatty components in a total amount by weight (G1b) and

one or more anionic surfactants in a total amount by weight (G4b),

characterized in that

the weight ratio (G1a)/(G1b) has a value of 0.8 to 1.2, preferably of 0.9 to 1.1, and

the weight ratio (G4a)/(G4b) has a value of 0.8 to 1.2, preferably of 0.9 to 1.1.

Preferred furthermore is a product for the oxidative coloring and/or lightening of keratinic fibers, whereby preparation (a) includes, based on its total weight,

one or more fatty components in a total amount by weight (G1a) and

one or more cationic surfactants in a total amount by weight (G3a), whereby preparation (b) includes, based on its total weight,

one or more fatty components in a total amount by weight (G1b) and

one or more cationic surfactants in a total amount by weight (G3b),

characterized in that

the weight ratio (G1a)/(G1b) has a value of 0.8 to 1.2, preferably of 0.9 to 1.1, and

the weight ratio (G3a)/(G3b) has a value of 0.8 to 1.2, preferably of 0.9 to 1.1.

Preferred furthermore is a product for the oxidative coloring and/or lightening of keratinic fibers, whereby preparation (a) includes, based on its total weight,

one or more anionic surfactants in a total amount by weight (G4a) and

one or more nonionic surfactants in a total amount by weight (G5a), whereby preparation (b) includes, based on its total weight,

one or more anionic surfactants in a total amount by weight (G4b) and

one or more nonionic surfactants in a total amount by weight (G5b),

characterized in that

the weight ratio (G4a)/(G4b) has a value of 0.8 to 1.2, preferably of 0.9 to 1.1, and

the weight ratio (G5a)/(G5b) has a value of 0.8 to 1.2, preferably of 0.9 to 1.1.

In the work leading to this invention, it has proven especially essential to match the particular content of C₁₂-C₃₀ fatty alcohols in preparations (a) and (b). The C₁₂-C₃₀ fatty alcohols are the most preferred fatty components.

Preferred, furthermore, is a product for the oxidative coloring and/or lightening of keratinic fibers, whereby

preparation (a) includes one or more fatty components from the group of C₁₂-C₃₀ fatty alcohols,

preparation (b) includes one or more fatty components from the group of C₁₂-C₃₀ fatty alcohols,

characterized in that
preparations (a) and (b) include the same fatty alcohols.

The term “same fatty alcohols” is understood to mean that preparations (a) and (b) both include the same fatty alcohol(s) or the same fatty alcohol raw material(s). For example, preparations (a) and (b) include the same fatty alcohols, if they both include cetearyl alcohols or both include Hydrenol D (cetearyl alcohol) or both include Lorol C12-C18 technical grade (mixture of C12-C18 fatty alcohols). If both preparations (a) and (b) include mixtures of fatty alcohols of different chain lengths, thus the distribution of the fatty alcohol chain lengths in both preparations must be the same. This is the case when the same raw materials are used in both preparations (a) and (b).

Preferred is a product for the oxidative coloring and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A), whereby

preparation (a) has a viscosity V1 of 2000 to 80,000 mPas (22° C./Brookfield viscometer/spindle 5/4 rpm),

preparation (a) includes one or more fatty components from the group of C₁₂-C₃₀ fatty alcohols,

(3) an aqueous cosmetic preparation (b) in chamber (B), whereby

preparation (b) has a viscosity V2 of 2000 to 80,000 mPas (22° C./Brookfield viscometer/spindle 5/4 rpm),

preparation (b) includes one or more fatty components from the group of C₁₂-C₃₀ fatty alcohols,

characterized in that

the ratio of the viscosities V1/V2 has a value of 0.3 to 3.0 and

preparations (a) and (b) include the same fatty alcohols.

The matching of preparations (a) and (b) with respect to their content of anionic surfactants has also proven beneficial for the emptying, homogeneity, and quantity consistency of the application mixture prepared from (a) and (b).

Also especially preferred is a product for the oxidative coloring and/or lightening of keratinic fibers, whereby

preparation (a) includes one or more anionic surfactants from the group of

physiologically acceptable salts of C₁₂-C₃₀ fatty acids and/or

physiologically acceptable salts of C₁₂-C₃₀ alkyl sulfates and/or

physiologically acceptable salts of C₁₂-C₃₀ alkyl ether sulfates with 2 to 30 ethylene oxide units, and

preparation (b) includes one or more anionic surfactants from the group of

physiologically acceptable salts of C₁₂-C₃₀ fatty acids and/or

physiologically acceptable salts of C₁₂-C₃₀ alkyl sulfates and/or

physiologically acceptable salts of C₁₂-C₃₀ alkyl ether sulfates with 2 to 30,

characterized in that
preparations (a) and (b) include the same anionic surfactants.

A furthermore especially preferred product for the oxidative coloring and/or lightening of keratinic fibers is characterized in that

preparation (a) includes one or more nonionic surfactants from the group of adducts to fatty alcohols with 2 to 30 mol of ethylene oxide per mole of fatty alcohol and

preparation (b) includes one or more nonionic surfactants from the group of adducts to fatty alcohols with 2 to 30 mol of ethylene oxide per mole of fatty alcohol characterized in that preparations (a) and (b) include the same nonionic surfactants.

Preparation (a) is preferably a formulation that is made alkaline and optionally includes oxidation dye precursors, if a color change is also desired in addition to the lightening. Preparation (b) is a formulation with oxidizing agents that is made acidic for reasons of stability.

In a further especially preferred embodiment, a product of the invention is therefore characterized in that

preparation (a) includes at least one alkalinizing agent, preferably ammonia and/or monoethanolamine, and

preparation (b) includes at least one oxidizing agent, preferably hydrogen peroxide.

Preparation (a) preferably includes at least one alkalinizing agent, preferably ammonia and/or monoethanolamine. Alkalinizing agents that can be used according to the invention can be selected from the group that is formed by ammonia, alkanolamines, basic amino acids, and inorganic alkalinizing agents such as alkali (alkaline earth) metal hydroxides, alkali (alkaline earth) metal metasilicates, alkali (alkaline earth) metal phosphates, and alkali (alkaline earth) metal hydrogen phosphates. Suitable inorganic alkalinizing agents are sodium hydroxide, potassium hydroxide, sodium silicate, and sodium metasilicate. Organic alkalinizing agents that can be used according to the invention can be selected from monoethanolamine, 2-amino-2-methylpropanol, and triethanolamine. The basic amino acids that can be used as alkalinizing agents according to the invention are preferably selected from the group formed by arginine, lysine, ornithine, and histidine, especially preferably arginine.

Ammonia (NH₃) in the form of its aqueous solution is customarily employed. Aqueous ammonia solutions include ammonia (NH₃) often in concentrations between 10% by weight and 32% by weight. Preferred in this case is the use of an aqueous ammonia solution, which includes 25% by weight of ammonia (NH₃).

Preferably, preparation (a) includes ammonia and/or monoethanolamine in amounts of 0.01 to 10% by weight, preferably of 0.1 to 7.5% by weight, more preferably of 0.2 to 5.5% by weight, and particularly preferably of 0.4 to 4.5% by weight, in each case based on the total weight of preparation (a).

Dyeing processes on keratin fibers typically take place in an alkaline environment. To treat keratin fibers and the skin as well as gently as possible, setting a too high pH is not desirable, however. It is preferable, therefore, if the pH of the ready-to-use agent (i.e., the mixture of preparations (a) and (b)) is between 7 and 11 and particularly between 8 and 10.5. pH values within the meaning of the present invention are pH values measured at a temperature of 22° C.

In this case, preparation (a) is preferably adjusted to an alkaline pH, and preparation (b) advantageously has an acidic pH.

In a further especially preferred embodiment, a product of the invention is characterized in that

preparation (a) has a pH of 8 to 12.5, preferably of 8.5 to 12, more preferably of 9 to 11.5, and particularly preferably of 9.5 to 11 and

preparation (b) has a pH of 1 to 7, preferably of 1.5 to 6, more preferably of 2.5 to 5.5, and particularly preferably of 3.0 to 5.0.

Preparations (a) and/or (b) can still include other additional ingredients.

If not just lightening but oxidative coloring as well are desired, preparation (a) can include in addition also one or more oxidation dye precursors, preferably at least one oxidation dye precursor of the developer type and at least one oxidation dye precursors of the coupler type. Especially suitable oxidation dye precursors of the developer type are thereby selected from at least one compound from the group, which is formed by p-phenylenediamine, p-toluylenediamine, 2-(2-hydroxyethyl)-p-phenylenediamine, 2-(1,2-dihydroxyethyl)-p-phenylenediamine, N,N-bis(2-hydroxyethyl)-p-phenylenediamine, 2-methoxymethyl-p-phenylenediamine, N-(4-amino-3-methylphenyl)-N-[3-(1H-imidazol-1-yl)-propyl]amine, N,N′-bis(2-hydroxyethyl)-N,N′-bis(4-aminophenyl)-1,3-diaminopropan-2-ol, bis(2-hydroxy-5-aminophenyl)methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo[1,2-a]pyrazol-1-one, and the physiologically acceptable salts thereof.

Especially suitable oxidation dye precursors of the coupler type in this case are selected from the group, formed by 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline, or mixtures of said compounds or the physiologically acceptable salts thereof.

In addition, preparation (a) can also include at least one direct dye from the group of anionic, nonionic and/or cationic dyes.

Especially preferably, this concerns one or more nonionic direct dyes from the group including HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9,1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

In another especially preferred embodiment, an agent of the invention is characterized in that it includes in addition one or more nonionic direct dyes from the group including HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9,1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol.

In addition, anionic direct dyes may also be present, which are known under the international names or trade names: Acid Yellow 1, Yellow 10, Acid Yellow 23, Acid Yellow 36, Acid Orange 7, Acid Red 33, Acid Red 52, pigment Red 57:1, Acid Blue 7, Acid Green 50, Acid Violet 43, Acid Black 1, Acid Black 52, bromophenol blue, and tetrabromophenol blue.

Suitable cationic direct dyes are cationic triphenylmethane dyes, such as, for example, Basic Blue 7, Basic Blue 26, Basic Violet 2, and Basic Violet 14, aromatic systems, substituted with a quaternary nitrogen group, such as, for example, Basic Yellow 57, Basic Red 76, Basic Blue 99, Basic Brown 16, and Basic Brown 17, cationic anthraquinone dyes, such as, HC Blue 16 (Bluequat B), and direct dyes, which include a heterocycle, which has at least one quaternary nitrogen atom, particularly Basic Yellow 87, Basic Orange 31, and Basic Red 51. The cationic direct dyes, which are marketed under the trademark Arianor, are also suitable cationic direct dyes according to the invention.

The oxidation dye precursors, i.e., developer components and coupler components, and the optional additional direct dyes can be used, for example, in an amount of 0.0001 to 5.0% by weight, preferably 0.001 to 3.5% by weight, in each case based on the total weight of preparation (a).

Preparations (a) and (b) furthermore can include additional active substances, auxiliary substances, and additives in order to improve the coloring or lightening performance and to set other desired properties of the agents.

It has proven advantageous, if the dyes (i.e., preparations (a) and particularly (b)), particularly if they include additional hydrogen peroxide, include at least one stabilizer or complexing agent. Especially preferred stabilizers are phenacetin, alkali benzoates (sodium benzoate), salicylic acid, and dipicolinic acid. Furthermore, all complexing agents in the state of the art can be used. Complexing agents preferred according to the invention are nitrogen-containing polycarboxylic acids, particularly EDTA and EDDS, and phosphonates, particularly 1-hydroxyethane-1,1-diphosphonate (HEDP), and/or ethylenediamine tetramethylene phosphonate (EDTMP), and/or diethylenetriamine pentamethylene phosphonate (DTPMP), or sodium salts thereof.

Further, the preparations of the invention can include other active substances, auxiliary substances, and additives, such as, for example, nonionic polymers such as, for example, vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or nonvolatile, straight-chain, branched or cyclic, crosslinked or noncrosslinked polyalkylsiloxanes (such as dimethicones or cyclomethicones), polyarylsiloxanes, and/or polyalkylarylsiloxanes, particularly polysiloxanes with organofunctional groups, such as substituted or unsubstituted amines (amodimethicones), carboxy, alkoxy, and/or hydroxyl groups (dimethicone copolyols), linear polysiloxanes(A)-polyoxyalkylene(B) block copolymers, grafted silicone polymers; cationic polymers such as quaternized cellulose ethers, polysiloxanes with quaternary groups, dimethyldiallylammonium chloride polymers, acrylamide-dimethyldiallylammonium chloride copolymers, dimethylaminoethylmethacrylate-vinylpyrrolidinone copolymers quaternized with diethylsulfate, vinylpyrrolidinone-imidazolinium-methochloride copolymers, and quaternized polyvinyl alcohol; zwitterionic and amphoteric polymers; anionic polymers such as, for example, polyacrylic acid or crosslinked polyacrylic acid; structurants such as glucose, maleic acid, and lactic acid, hair-conditioning compounds such as phospholipids, for example, lecithin and kephalins; perfume oils, dimethyl isosorbide, and cyclodextrins; fiber-structure-improving active substances, particularly mono-, di-, and oligosaccharides such as, for example, glucose, galactose, fructose, fruit sugar, and lactose; dyes for coloring the agent; antidandruff agents such as piroctone olamine, zinc omadine, and climbazole; amino acids and oligopeptides; protein hydrolysates with an animal and/or vegetable base, and in the form of their fatty acid condensation products or optionally anionically or cationically modified derivatives; vegetable oils; light stabilizers and UV blockers; active substances such as panthenol, pantothenic acid, pantolactone, allantoin, pyrrolidinone carboxylic acids, and salts thereof, as well as bisabolol; polyphenols, particularly hydroxycinnamic acids, 6,7-dihydroxycoumarins, hydroxybenzoic acids, catechins, tannins, leukoanthocyanidins, anthocyanidins, flavanones, flavones, and flavonols; ceramides or pseudoceramides; vitamins, provitamine, and vitamin precursors; plant extracts; fats and waxes such as fatty alcohols, beeswax, montan wax, and paraffins; swelling and penetration agents such as glycerol, propylene glycol monoethyl ether, carbonates, hydrogen carbonates, guanidines, ureas, and primary, secondary, and tertiary phosphates; opacifiers such as latex, styrene/PVP and styrene/acrylamide copolymers; pearlescent agents such as ethylene glycol mono- and distearate and PEG-3 distearate; pigments and propellants such as propane-butane mixtures, N₂O, dimethyl ether, CO₂, and air.

The selection of these additional substances is made by the skilled artisan according to the desired properties of preparations (a) and (b). In regard to other facultative components and the employed amounts of said components, reference is made expressly to relevant handbooks known to the skilled artisan. The additional active and auxiliary substances are used in the agents of the invention preferably in amounts in each case of 0.0001 to 25% by weight, particularly of 0.0005 to 15% by weight, based on the total weight of the application mixture.

The dispenser within the meaning of the present invention is understood to mean a container including at least two chambers ((A) and (B)), which can be fabricated, for example, from plastic, metal, and/or plastic-metal composites. The dispenser can have, inter alia, the shape of a box, a bottle, a container, a pressurized container, or a tube. Suitable for the product of the invention are all dispenser designs that assure that the product compositions (a) and (b) present in both chambers of the dispenser are kept reliably isolated from another until removal.

Preferably, the individual product compositions are combined to form a total formulation during the product applications, in order to exploit in this way the full effectiveness of the total formulation and to make product use easier in general. For this purpose, the dispenser expediently includes, in addition to chambers (A), (B), a dispensing head within which the product compositions (a), (b) are conveyed from the chambers to outlet opening (C). In this regard, a suitable mixing device is formed in the dispensing head; upstream from outlet opening (C), said device provides for the desired mixing of the product compositions (a), (b), before the mixed total formulation, i.e., (a)+(b), is dispensed via outlet opening (C). For example, a generic dispenser with a comparable mixing device is known from DE 3729491 A1, whereby the mixing device therein only has a very short mixing section, however. Such a mixing device according to the invention is structurally integrated directly into the dispensing head or alternatively, however, disposed as a separate structural element within the dispensing head. Static mixers or comparably acting mixing sections, for example, are to be understood to mean suitable mixing devices within the meaning of the invention; these are traversed by the free-flowing product compositions (a), (b) and sufficiently mixed during this through-flow. To this end, such a mixing section typically has suitable flow baffles or flow spoilers, which cause mixing of individual fluid components due to generated turbulences during the surround-flow. Critical for the quality of the mixing of individual fluid components, in this case the product compositions (a), (b), within the mixing device is, inter alia, the selective coordination of the length of the mixing section and the design of the flow baffles to the rheological properties of the product compositions (a), (b). Defined minimum lengths of the mixing section are usually necessary for an adequate mixing result of the mixing device of the invention. Therefore, the mixing section of the mixing device of the invention is preferably designed such that a defined minimum length of the mixing section is guaranteed, without abandoning an overall compact construction of the mixing device and thus of the dispensing head. Thus, adequate mixing results are guaranteed and simultaneously the desired compact outer dimensions of the mixing device and of the dispensing head are assured. To this end, the mixing section is formed, for example, spiral-shaped or comparably compact within the dispensing head.

Chamber (B) of the dispenser can be disposed beside, above, or below chamber (A). Furthermore, chamber (B) can lie within chamber (A) or chamber (A) can lie within chamber (B), whereby in each case the one chamber encloses the other chamber partially or completely.

The capacity of chambers (A) and (B) can be 10 cm³ to 1000 cm³ in each case, and the capacity of chambers (A) and (B) can be the same or different. Preferably, the capacity of chambers (A) and (B) is the same. Preferably, chambers A and B are arranged next to one another.

The dispenser has an outlet opening (C), which is in fluid communication with both chambers (A) and (B) or can be brought into fluid communication with it via valves.

When the application composition is removed, both formulations (a) and (b) are removed simultaneously via the common outlet opening and are preferably already mixed together upstream by means of the above-described mixing device. The usually reactive individual formulations (a) and (b) thereby come into contact for the first time during product use and hereby form the ready-to-use mixture.

Outlet opening (C) can be, for example, a pressurized container-dispensing device, a pump element, a valve, or a dispensing device for pasty materials. The removal of preparations (a) and (b) via the common outlet opening (C) can occur by pressing the valve or by pumping the pump element.

The dispenser can also be a squeeze container; in this case, preparations (a) and (b) are removed preferably by pressing the squeeze container, whereby the pressure on the squeeze container results in the discharging of preparations (a) and (b) from the common outlet opening (C).

Furthermore, all designs of an outlet opening (C) that enable formulations (a) and (b) to leave the dispenser together are also conceivable.

An especially preferred product is characterized in that

the dispenser is a pressurized container

outlet opening (C) is a pressurized container-dispensing device, which also has an appropriate mixing device, and that

the dispenser in addition includes at least one propellant from the group including propane, propene, n-butane, isobutane, isobutene, n-pentane, pentene, isopentane, isopentene, air, nitrogen, argon, N₂O, and/or CO₂.

In the aforementioned preferred embodiment, the product of the invention includes a pressurized container. Vessels made of metal (aluminum, tin plate, tin), protected or non-splintering plastic or of glass that is coated on the outside with plastic may be used as compressed-gas containers; pressure and breaking strength, corrosion resistance, easy fillability, as well as aesthetic aspects, handling, printability, etc., play a role in their selection. Special protective interior coatings can assure corrosion resistance against the preparation within the pressurized container if necessary.

Very especially preferably, the two chambers (A) and (B) separated from one another are two deformable pouches, preferably made of laminated aluminum, which are each in communication with outlet opening (C). Outlet opening (C) is a pressurized container-dispensing device. Both pouches are in a box-shaped pressurized container, whereby the pressurized container together with the pressurized container-dispensing device close off the dispenser pressure-tight outwardly. The space between the outer wall of the pouch and the inner wall of the pressurized container is filled with at least one propellant. Suitable dispensers are known, for example, from US 2009/0108021 A1. A very especially preferred product for treating keratinic fibers is therefore characterized by such a dispenser with a suitable pressurized container, which has the above-described mixing device. A dispenser constructed in this way takes into account not only the chemical reactivity of the employed individual product compositions (a), (b), but moreover as a result of the mixing provides for a high effectiveness as well of the employed total formulation. Especially good effects according to the invention are achieved when the internal pressure of the pressurized container is at least 1.8 bar, particularly at least 2.5 bar.

The product furthermore includes a dispensing device (corresponding to outlet opening C), which can have a valve for discharging the application mixture. In a preferred embodiment of the invention, the valve has a valve disc with a coating or a polymeric plastic coating and just such a flexible element with a reset function, which after the operation ends resets the valve to the closed position (=neutral position of the valve). Corresponding cosmetic products in which the aerosol dispensing device includes a valve, which has a valve cone and/or a flexible element with reset function, which is/are covered with a coating or a polymeric plastic, are also preferred according to the invention.

In another preferred embodiment of the invention, the valve has a flexible element with a reset function and/or a valve disc made of at least one plastic, preferably a elastomeric plastic. Here as well, cosmetic products of the invention in which the valve has a flexible element with a reset function and/or a valve cone made of at least one plastic are preferred, whereby preferred plastics are elastomeric plastics. Especially preferred elastomeric plastics are selected from Buna, particularly Buna N, Buna 421, Buna 1602, and Buna KA 6712, neoprene, butyl, and chlorobutyl.

In another preferred embodiment of the invention, the flexible element with a reset function can be formed as a spiral spring or helical compression spring. In another preferred embodiment of the invention, the flexible element of the valve with the reset function can be formed integrally with the valve cone and have flexible legs. Such a spring can be made of metal or plastic.

All valves used according to the invention preferably have an internally coated valve disc, whereby the coating and valve material are compatible with one another. If aluminum valves are used according to the invention, thus the valve discs thereof can be coated on the inside, e.g., with a Micoflex coating. If tin plate valves are used according to the invention, thus the valve discs thereof can be coated on the inside, e.g., with PET (polyethylene terephthalate). The employed containers, which can be made, e.g., of tin plate or aluminum, aluminum containers being preferred according to the invention, must also be painted or coated on the interior because of the corrosivity of the water-in-oil emulsions, used as taught by the invention.

If the product of the invention is applied via a pressurized container, the dispensers include in addition at least one propellant from the group including propane, propene, n-butane, isobutane, isobutene, n-pentane, pentene, isopentane, isopentene, air, nitrogen, argon, N₂O, and/or CO₂. Within this group, the permanent gases: air, nitrogen, argon, N₂O, and/or CO₂ are preferred; nitrogen, argon, and/or CO₂ are very especially preferred.

Furthermore, it has proven to be preferable if the propellants are also kept at specific pressures in the dispenser. In a preferred embodiment, the dispensers of the invention include one or more additional propellants therefore with a pressure of 3 to 12 bar, preferably of 4 to 10 bar, and especially preferably of 5 to 8 bar, in each case based on the pressure of the propellants between the pouch-shaped chambers (A) and (B) and the pressurized container.

In another especially preferred embodiment, the dispenser is a dispenser for pasty materials, a multi-chamber tube, or a squeeze container.

In another very especially preferred embodiment, a product of the invention is characterized in that

the dispenser is a dispenser for pasty materials, a tube, or a squeeze tube and

outlet opening (C) is a pump element, a valve, or a dispensing device for pasty materials.

The product of the invention according to an alternative design form includes a multi-chamber tube, which has an inner and outer chamber, both of which end in a common outlet opening (C). The head area is designed such that the two preparations leave the tube together as soon as pressure is exerted on them. The design of said head region determines the pattern in which the preparations leave the tube. In principle, the present invention can comprise any distribution of chambers within the tube. In a first embodiment, for example, the two individual chambers can be arranged next to one another in an outer shell. In an embodiment especially preferred according to the invention, the multi-chamber tube, however, consists of an inner tube completely surrounded by an outer tube.

The multi-chamber tube is preferably fabricated of a material that is suitable for packaging oxidative color changing agents of this type. Laminated aluminum has proven especially suitable according to the invention both for the outer walls and for the inner walls. However, tubes made of plastic laminate (PE, PET, PP) or plastic coextrudates (PE, PET, PP) are also conceivable. Moreover, in one embodiment the material of the inner tube can be selected independently of the material of the outer tube. A tube in which the inner tube is fabricated of an aluminum laminate, which optionally is also protected with a coating, and the outer tube either of aluminum laminate or plastic laminate has proven very especially preferable according to the invention. Aluminum laminate according to the invention is understood to mean a plastic-coated aluminum layer.

It is especially advantageous if the shoulder region of the outer tube is reinforced with round blanks which have especially good barrier properties. In this regard, it is advantageous to incorporate aluminum into the material of the round blanks. To prevent the escape of the mixture during storage and to assure the consumer of the intactness of the tube, it is advantageous to seal the outlet opening with a tamper-evident closure made of aluminum or plastic which is removed by the user.

The selection of the volumes of the individual chambers (A) and (B) is determined by the desired ratio of the volumes of preparation (a) and preparation (b). The volumes of chambers (A) and (B) are preferably the same. The quantity ratio of preparation (a) to the amount of preparation (b) according to the invention is preferably in a range of 1:3 to 3:1; a range of 1:1.5 to 1.5:1 is preferred according to the invention, and a quantity ratio of 1:1 is especially preferred.

Likewise, very especially preferred is a product for the oxidative dyeing and/or lightening of keratinic fibers, including

(1) a dispenser, which

has two chambers (A) and (B) separate from one another,

has an outlet opening (C), which is in communication with chamber (A) and with chamber (B),

(2) an aqueous cosmetic preparation (a) in chamber (A) having a viscosity V1 of 1000 to 100,000 mPas (22° C./Brookfield viscometer/spindle 5/4 rpm),
(3) an aqueous cosmetic preparation (a) in chamber (B) having a viscosity V2 of 1000 to 100,000 mPas (22° C./Brookfield viscometer/spindle 5/4 rpm),
characterized in that

the ratio of the viscosities V1/V2 has a value of 0.3 to 3.0 and

the weight ratio of the total weight of preparation (a) to the total weight of preparation (b) is a value of 0.5 to 2.0, preferably of 0.6 to 1.8, more preferably of 0.7 to 1.4, and particularly preferably of 0.8 to 1.2.

The indicated viscosities are viscosities that were measured at 22° C. (22° C./Brookfield viscometer/spindle 5/4 rpm).

The products of the invention can be used in methods for the oxidative changing of hair color. These methods distinguish themselves for the consumer by the particular ease of use, because there are no time-consuming or error-prone steps for preparation of the application mixtures by the consumer. In addition, a particularly uniform color result is enabled with use of the products, because due to the special rheological matching of formulations (a) and (b) it is possible to remove the two formulations in precisely defined proportions, which are always the same relative to one another, during the entire application process.

The removal of the application mixture in this case usually occurs in batches, so that chambers (A) and (B) of the product are emptied stepwise in a defined ratio to one another. The consumer generally removes the amount of the application mixture, necessary for dyeing or lightening one or more strands or sections of hair, from the dispenser, and then applies this amount to the hair. This process is repeated by the consumer until either the product has been totally emptied or the consumer has treated all of the hair.

In particular, a continuous removal of the product is also conceivable in the packaging of the product in the form of an aerosol, however; i.e., in this case chambers (A) and (B) are emptied continuously by the operation of outlet opening (C), whereby here as well the composition of the application mixture (of preparations (a) and (b)) is the same and defined at each point in time of the removal.

A further subject matter of the present invention is a method for coloring and/or lightening of keratinic fibers during use of a product of the first subject matter of the invention, whereby

the dispenser is emptied continuously in one step or in batches in individual steps and

in each step defined, constant amounts of preparations (a) and (b) are removed each time from chambers (A) and (B) via the common outlet opening (C) of the dispenser until both chambers (A) and (B) have been emptied completely.

A method is preferred in which chambers (A) and (B) are emptied in batches in a plurality of steps.

Especially preferred is a method for coloring and/or lightening of keratinic fibers during use of a product of the first subject matter of the invention, whereby

the dispenser is emptied in batches in a plurality of steps and

in each step defined, constant amounts of preparations (a) and (b) are removed each time from chambers (A) and (B) via the common outlet opening (C) of the dispenser until both chambers (A) and (B) have been emptied completely.

It can be desirable in this regard that the same amounts of preparations (a) and (b) are removed in each step or each time. If one of chambers (A) or (B) has a larger volume or capacity, however, and/or if one of preparations (a) and (b) is present in a higher amount in the product, thus it can also be desirable to remove preparations (a) and (b) in each step in a constant quantity ratio of 3:1 to 1:3.

It is especially preferred if the amount of preparations (a) and (b), removed in steps, is the same, i.e., if the same amounts (a) and (b) are removed from the dispenser in each removal step.

A method for coloring and/or lightening of keratinic fibers, therefore, is very especially preferred, which is characterized in that

in each step the same amounts of preparations (a) and (b) are removed each time from chambers (A) and (B) via the common outlet opening (C) of the dispenser until both chambers (A) and (B) have been emptied completely.

The statements made regarding the product of the invention apply mutatis mutandis with respect to the other preferred embodiments of the method of the invention.

### EXAMPLES

### 1. Preparation of Preparations (a) and (b)

### Example 1

The following preparations (a) and (b) were prepared (all quantities are given in % by weight of active substance):

| | | Preparation | Preparation |
|---|---|---|---|
| Ingredients | | (a) | (b) |
| Lanette D (cetearyl alcohol) | Fatty component | 3.30 | 3.30 |
| Lorol C12-C18 (techn.) (C12-C18 | Fatty component | 1.20 | 1.20 |
| fatty alcohols) | | | |
| Glyceryl oleate | Fatty component | 0.6 | 0.6 |
| Coco-glucosides | Nonionic. emulsifier | 0.6 | 0.6 |
| Ceteareth-20 | Nonionic emulsifier | 0.30 | 0.30 |
| Ceteareth-12 | Nonionic emulsifier | 0.30 | 0.30 |
| Sodium myreth sulfate (C14 fatty | Anionic surfactant | 1.96 | 1.96 |
| alcohol, ethoxylated with 3 EO, | | | |
| sulfate, sodium salt) | | | |
| Sodium myreth sulfate (C14 fatty | Anionic surfactant | 1.96 | 1.96 |
| alcohol, ethoxylated with 3 EO, | | | |
| sulfate, sodium salt) | | | |
| Xanthan gum | Thickening polymer | 1.00 | 1.00 |
| Propylene glycol | Solvent | 2.00 | 2.00 |
| p-Toluylenediamine, sulfate | ODP | 0.111 | — |
| Resorcinol | ODP | 0.03 | — |
| m-Aminophenol | ODP | 0.002 | — |
| 4-Chlororesorcinol | ODP | 0.03 | — |
| 2-Methylresorcinol | ODP | 0.01 | — |
| Ammonium sulfate (NH₄)₂SO₄ | Salt | 0.93 | — |
| Sodium sulfite Na₂SO₃ | Salt | 0.40 | — |
| Vitamin C | Reducing agent | 0.10 | — |
| Dipicolinic acid | Stabilizer | 0.10 | — |
| Disodium pyrophosphate | Stabilizer | — | 0.91 |
| Hydroxyethane-1,1-diphosphonic | Stabilizer | 0.20 | 0.90 |
| acid | | | |
| Ammonia (NH3) | Alkalinizing agent | 2.00 | — |
| Hydrogen peroxide | OX | — | 6.00 |
| Water | | To 100 | To 100 |
| Viscosity | | 20,000 mPas | 20,000 mPas |
| (22° C./Brookfield viscometer/ | | | |
| spindle 5/4 rpm) | | | |

### Products and Abbreviations:

[1] Product W 37194: 1-Propanaminium, N,N,N-trimethyloxo-2-propenyl)amino]-, chloride, polymer with sodium 2-propenoate (INCI: ACRYLAMIDOPROPYLTRIMONIUM CHLORIDE/ACRYLATES COPOLYMER)
(20% aqueous solution)
ODP: oxidation dye precursors
OX: oxidizing agent

### Example 2

The following preparations (a) and (b) were prepared (all quantities are given in % by weight of active substance):

| | | Preparation | Preparation |
|---|---|---|---|
| Ingredients | | (a) | (b) |
| Lanette D (cetearyl alcohol) | Fatty component | 3.30 | 3.30 |
| Lorol C12-C18 (techn.) (C12-C18 | Fatty component | 1.20 | 1.20 |
| fatty alcohols) | | | |
| Glyceryl oleate | Fatty component | 0.6 | 0.6 |
| Coco-glucosides | Nonionic emulsifier | 0.6 | 0.6 |
| Ceteareth-20 | Nonionic emulsifier | 0.30 | 0.30 |
| Ceteareth-12 | Nonionic emulsifier | 0.30 | 0.30 |
| Sodium acrylate, | Thickening polymer | 1.00 | 1.00 |
| trimethylammoniopropylacrylamide | | | |
| chloride, copolymer | | | |
| Sodium myreth sulfate (C14 fatty | Anionic surfactant | 1.96 | 1.96 |
| alcohol, ethoxylated with 3 EO, | | | |
| sulfate, sodium salt) | | | |
| Xanthan Gum | Thickening polymer | 1.00 | 1.00 |
| Propylene glycol | Solvent | 2.00 | 2.00 |
| p-Toluylenediamine, sulfate | ODP | 0.111 | — |
| Resorcinol | ODP | 0.03 | — |
| m-Aminophenol | ODP | 0.002 | — |
| 4-Chlororesorcinol | ODP | 0.03 | — |
| 2-Methylresorcinol | ODP | 0.01 | — |
| Ammonium sulfate (NH₄)₂SO₄ | Salt | 0.93 | — |
| Sodium sulfite Na₂SO₃ | Salt | 0.40 | — |
| Vitamin C | Reducing agent | 0.10 | — |
| Dipicolinic acid | Stabilizer | — | 0.10 |
| Disodium pyrophosphate | Stabilizer | — | 0.91 |
| Hydroxyethane-1,1-diphosphonic | Stabilizer | 0.20 | 0.90 |
| acid | | | |
| Ammonia (NH3) | Alkalinizing agent | 2.00 | — |
| Hydrogen peroxide | OX | — | 6.00 |
| Water | | To 100 | To 100 |
| Viscosity | | 20,000 mPas | 20,000 mPas |
| (22° C./Brookfield viscometer/ | | | |
| spindle 5/4 rpm) | | | |

### Example 3

The following preparations (a) and (b) were prepared (all quantities are given in % by weight of active substance):

| | | Preparation | Preparation |
|---|---|---|---|
| Ingredients | | (a) | (b) |
| Lanette D (cetearyl alcohol) | Fatty component | 3.3 | 3.3 |
| Lorol C12-C18 (techn.) (C12-C18 | Fatty component | 1.20 | 1.20 |
| fatty alcohols) | | | |
| Ceteareth-20 | Nonionic emulsifier | 0.30 | 0.30 |
| Ceteareth-12 | Nonionic emulsifier | 0.30 | 0.30 |
| Coco glucosides | Nonionic emulsifier | 1.00 | 1.00 |
| Sodium laureth-6-carboxylate | Anionic surfactant | 5.00 | 5.00 |
| Sodium myreth sulfate (C14 fatty | Anionic surfactant | 0.98 | 0.98 |
| alcohol, ethoxylated with 3 EO, | | | |
| sulfate, sodium salt) | | | |
| 1-(2-Hydroxyethyl)-4,5- | ODP | 1.90 | — |
| diaminopyrazole, sulfate | | | |
| m-Aminophenol | ODP | 0.3 | — |
| 2-Methylresorcinol | ODP | 0.05 | — |
| 5-Amino-2-methylphenol | ODP | 0.80 | — |
| Ammonium sulfate (NH₄)₂SO₄ | Salt | 0.41 | — |
| Sodium sulfite Na₂SO₃ | Salt | 0.40 | — |
| Vitamin C | Reducing agent | 0.10 | — |
| Potassium hydroxide | Alkalinizing agent | 0.40 | 0.45 |
| Hydroxyethane-1,1-diphosphonic | Stabilizer | 0.2 | 1.5 |
| acid | | | |
| Propylene glycol | Solvent | 2.0 | 2.0 |
| Xanthan gum | Thickening polymer | 1.20 | 1.00 |
| Sodium acrylate, | Thickening polymer | 1.00 | 1.00 |
| trimethylammoniopropylacrylamide | | | |
| chloride, copolymer | | | |
| Ammonia (NH3) | Alkalinizing agent | 1.63 | — |
| Dipicolinic acid | Stabilizer | — | 0.10 |
| Disodium pyrophosphate | Stabilizer | — | 0.03 |
| Hydrogen peroxide | OX | — | 6.10 |
| Water | | To 100 | To 100 |
| Viscosity | | 17,000 | 18,000 |
| (22° C./Brookfield viscometer/ | | | |
| spindle 5/4 rpm) | | | |

### Example 4

The following preparations (a) and (b) were prepared (all quantities are given in % by weight of active substance):

| | | Preparation | Preparation |
|---|---|---|---|
| Ingredients | | (a) | (b) |
| Lanette D (cetearyl alcohol) | Fatty component | 4.95 | 4.95 |
| Lorol C12-C18 (techn.) (C12-C18 | Fatty component | 1.80 | 1.80 |
| fatty alcohols) | | | |
| Ceteareth-20 | Nonionic emulsifier | 0.45 | 0.45 |
| Ceteareth-12 | Nonionic emulsifier | 0.45 | 0.45 |
| Coco-glucosides | Nonionic emulsifier | 1.00 | 1.00 |
| Sodium laureth-6-carboxylate | Anionic surfactant | 5.00 | 5.00 |
| Sodium myreth sulfate (C14 fatty | Anionic surfactant | 0.98 | 0.98 |
| alcohol, ethoxylated with 3 EO, | | | |
| sulfate, sodium salt) | | | |
| p-Toluylenediamine, sulfate | ODP | 2.20 | — |
| 2-Amino-4-[(2- | ODP | 0.05 | — |
| hydroxyethyl)amino]-anisol, sulfate | | | |
| Resorcinol | ODP | 0.80 | — |
| m-Aminophenol | ODP | 0.30 | — |
| Sodium sulfite Na₂SO₃ | Salt | 0.40 | — |
| Vitamin C | Reducing agent | 0.10 | — |
| Hydroxyethane-1,1-diphosphonic | Stabilizer | 0.20 | 1.50 |
| acid | | | |
| Propylene glycol | Solvent | 3.00 | 3.00 |
| Xanthan gum | Thickening polymer | 1.50 | 1.50 |
| Sodium acrylate, | Thickening polymer | 1.00 | 1.00 |
| trimethylammoniopropylacrylamide | | | |
| chloride, copolymer | | | |
| Ammonia (NH3) | Alkalinizing agent | 1.63 | — |
| Dipicolinic acid | Stabilizer | — | 0.1 |
| Disodium pyrophosphate | Stabilizer | — | 0.03 |
| Potassium hydroxide | Alkalinizing agent | 1.80 | 0.91 |
| Hydrogen peroxide | OX | — | 6.1 |
| Water | | To 100 | To 100 |
| Viscosity | | 42,3000 | 43,000 |
| (22° C./Brookfield viscometer/ | | | |
| spindle 5/4 rpm) | | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A product for the oxidative coloring and/or lightening of keratinic fibers, including
(1) a dispenser, which
has two chambers (A) and (B) separate from one another, and
has an outlet opening (C), which is in communication with chamber (A) and with chamber (B),
(2) an aqueous cosmetic preparation (a) in chamber (A) having a viscosity V1 of 1,000 to 100,000 mPas at 22° C. using a Brookfield viscometer/spindle 5/4 rpm,
(3) an aqueous cosmetic preparation (a) in chamber (B) having a viscosity V2 of 1,000 to 100,000 mPas at 22° C. using a Brookfield viscometer/spindle 5/4 rpm,
wherein the ratio of the viscosities V1/V2 has a value of 0.3 to 3.0.

2. The product according to claim 1, wherein
preparation (a) has a viscosity of 2,000 to 80,000 mPas at 22° C. using a Brookfield viscometer/spindle 5/4 rpm, and
preparation (b) has a viscosity of 2,000 to 80,000 mPas at 22° C. using a Brookfield viscometer/spindle 5/4 rpm.

3. The product according to claim 1, wherein the ratio of the viscosities V1/V2 has a value of 0.5 to 2.0.

4. The product according to claim 1, wherein at least 70% by weight of the ingredients, included in preparations (a) and (b), are the same.

5. The product according to claim 1, wherein preparation (a) includes, in each case based on its total weight,
one or more fatty components in a total amount by weight (G1a) and/or
one or more thickening polymers in a total amount by weight (G2a) and/or
one or more cationic surfactants in a total amount by weight (G3a) and/or
one or more anionic surfactants in a total amount by weight (G4a) and/or
one or more nonionic surfactants in a total amount by weight (G5a) and/or
one or more zwitterionic surfactants in a total amount by weight (G6a) and/or,
whereby preparation (b) includes, in each case based on its total weight,
one or more fatty components in a total amount by weight (G1b) and/or
one or more thickening polymers in a total amount by weight (G2b) and/or
one or more cationic surfactants in a total amount by weight (G3b) and/or
one or more anionic surfactants in a total amount by weight (G4b) and/or
one or more nonionic surfactants in a total amount by weight (G5b) and/or
one or more zwitterionic surfactants in a total amount by weight (G6b),
wherein
at least one of the weight ratios from the group consisting of (G1a)/(G1b), (G2a)/(G2b), (G3a)/(G3b), (G4a)/(G4b) (G5a)/(G5b), and (G6a)/(G6b) have a value of 0.8 to 1.2.

6. The product according to claim 5, wherein the ratio of the sum of the total amounts by weight G1a, G2a, G3a, G4a, G5a, and G6a in preparation (a) to the sum of the total amounts by weight G1b, G2b, G3b, G4b, G5b and G6b in preparation (b), i.e., the ratio
[(G1a)+(G2a)+(G3a)+(G4a)+(G5a)+(G6a)]/[(G1b)+(G2b)+(G3b)+(G4b)+(G5b)+(G6b)]
has a value of 0.6 to 1.4.

7. The product according to claim 1, wherein preparation (a) includes, in each case based on its total weight,
one or more fatty components in a total amount by weight (G1a) and
one or more anionic surfactants in a total amount by weight (G4a),
wherein preparation (b) includes, based on its total weight,
one or more fatty components in a total amount by weight (G1b) and
one or more anionic surfactants in a total amount by weight (G4b),
and wherein
the weight ratio (G1a)/(G1b) has a value of 0.8 to 1.2, and
the weight ratio (G4a)/(G4b) has a value of 0.8 to 1.2.

8. The product according to claim 1, wherein preparation (a) includes, based on its total weight,
one or more fatty components in a total amount by weight (G1a) and
one or more cationic surfactants in a total amount by weight (G3a),
wherein preparation (b) includes, based on its total weight,
one or more fatty components in a total amount by weight (G1b) and
one or more cationic surfactants in a total amount by weight (G3b),
and wherein
the weight ratio (G1a)/(G1b) has a value of 0.8 to 1.2, and
the weight ratio (G3a)/(G3b) has a value of 0.8 to 1.2.

9. The product according to claim 1, wherein preparation (a) includes
one or more anionic surfactants in a total amount by weight (G4a) and
one or more nonionic surfactants in a total amount by weight (G5a),
wherein preparation (b) includes
one or more anionic surfactants in a total amount by weight (G4b) and
one or more nonionic surfactants in a total amount by weight (G5b),
and wherein
the weight ratio (G4a)/(G4b) has a value of 0.8 to 1.2, and
the weight ratio (G5a)/(G5b) has a value of 0.8 to 1.2.

10. The product according to claim 1, wherein
preparation (a) includes one or more fatty components from the group of C₁₂-C₃₀ fatty alcohols, and
preparation (b) includes one or more fatty components from the group of C₁₂-C₃₀ fatty alcohols,
wherein preparations (a) and (b) include the same fatty alcohols.

11. The product according to claim 1, wherein
preparation (a) includes one or more anionic surfactants from the group consisting of
physiologically acceptable salts of C₁₂-C₃₀ fatty acids,
physiologically acceptable salts of C₁₂-C₃₀ alkyl sulfates, and
physiologically acceptable salts of C₁₂-C₃₀ alkyl ether sulfates with 2 to 30 ethylene oxide units, and
preparation (b) includes one or more anionic surfactants from the group consisting of
physiologically acceptable salts of C₁₂-C₃₀ fatty acids,
physiologically acceptable salts of C₁₂-C₃₀ alkyl sulfates, and
physiologically acceptable salts of C₁₂-C₃₀ alkyl ether sulfates with 2 to 30 ethylene oxide units,
and wherein preparations (a) and (b) include the same anionic surfactants.

12. The product according to claim 1, wherein
preparation (a) includes one or more nonionic surfactants from the group of adducts to fatty alcohols with 2 to 30 mol of ethylene oxide per mole of fatty alcohol and
preparation (b) includes one or more nonionic surfactants from the group of adducts to fatty alcohols with 2 to 30 mol of ethylene oxide per mole of fatty alcohol
and wherein preparations (a) and (b) include the same nonionic surfactants.

13. The product according to claim 1, wherein
preparation (a) has a pH of 8 to 12.5, and
preparation (b) has a pH of 1 to 7.

14. The product according to claim 1, wherein
the dispenser is an aerosol pressurized container,
outlet opening (C) is an aerosol dispensing device, and
preparations (a) and (b) in each case include in addition at least one propellant from the group consisting of propane, propene, n-butane, isobutane, isobutene, n-pentane, pentene, isopentane, isopentene, air, nitrogen, argon, N₂O, and/or CO₂.

15. The product according to claim 1, wherein
the dispenser is a dispenser for pasty materials, a tube, or a squeeze tube, and
outlet opening (C) is a pump element, a valve, or a dispensing device for pasty materials.
